(19) United States
(12) Patent Application Publication
(10) Pub. No.: US 2026/0043809 A1
(43) Pub. Date: Feb. 12, 2026

(54) AFFINITY CAPTURE REAGENTS FOR MASS SPECTROMETRY

(71) Applicant: OXFORD UNIVERSITY INNOVATION LIMITED, Oxford (GB)

(72) Inventors: Carol ROBINSON, Oxford Oxfordshire (GB); Corinne LUTOMSKI, Oxford Oxfordshire (GB); Tarick EL-BABA, Oxford Oxfordshire (GB)

(21) Appl. No.: 19/144,774

(22) PCT Filed: Dec. 28, 2023
(86) PCT No.: PCT/GB2023/053381

§ 371 Date: Jun. 30, 2025

(30) Foreign Application Priority Data
Dec. 30, 2022 (GB) 2219855.0

(51) Int. Cl. G01N 33/68

(52) U.S. Cl. CPC G01N 33/6848; G01N 33/6821; G01N 33/6824; G01N 33/6845

(57) ABSTRACT

The invention relates to a peptide mass tag for recovery and identification of target proteins by mass spectrometry. The invention further relates to fusion polypeptides including the peptide mass tag and a method of analysing a target protein comprising use of the peptide mass tag.

### FIELD OF THE INVENTION

The invention relates to a peptide mass tag for recovery and identification of target proteins by mass spectrometry. A plurality of such tags may be provided for multiplex applications, for example in a kit or library. The invention also relates to a polypeptide comprising a target protein or an affinity ligand for a target protein fused to said tag, and a composition comprising said affinity ligand in complex with a target protein. The invention also relates to a nucleic acid or vector encoding the peptide mass tag or a polypeptide fused to the peptide mass tag, and a host cell comprising such a nucleic acid or vector. The invention further relates to a method of analysing a target protein comprising use of the peptide mass tag.

### BACKGROUND OF THE INVENTION

Native mass spectrometry (MS) has emerged as the premier biophysical technique to monitor protein-protein and protein-ligand interactions by monitoring changes in mass. As it is readily capable of deciphering such changes with near perfect mass accuracy, native MS has been complemented by advancements in structural biology by enabling for direct correlations between structure, composition, and function. Recent studies have illustrated that native MS can be used to decipher such compositional changes directly from signaling complexes embedded in native cell membranes—an exciting step towards understanding biological processes from as close to a biological context as possible.ⁱ

It is becoming increasingly attractive to use native mass spectrometry to study molecular changes in context, that is, endogenous proteins in complex with their interactors, directly from or as close to their native environments as possible. Techniques for structural studies of endogenous proteins by CryoEM have been developed,^{ii,iii} and further technological developments to enrich or label endogenous biomolecules by leveraging interactions with monoclonal antibodies, DNA origamis,^{iv} and modifying genomes to couple tags^{v} are emerging. Such advancements have enabled for the enrichment and/or isolation of endogenous proteins and protein complexes for detailed structural studies that simply cannot be recapitulated in vitro. However, these tools are challenging to adapt to living organisms, and, are highly specialized.

As an added layer of complexity, accurate mass analysis using high resolution mass spectrometers has become increasingly scrutinized as a means for bona fide assignment of endogenous biomolecules. Thus, tandem mass spectrometry is essential for molecular identification. In tandem MS, the peptide backbone of intact molecules is broken to produce strings of amino acid fragments that are detected by the mass spectrometer which are then used to identify molecular sequences within lists of candidate proteins. However, fragmentation of native, folded proteins is inherently inefficient and often leads to the generation of few fragments (fewer than acceptable for statistical validation of candidate protein sequences). For complex samples (such as cell lysates) where many different proteins and protein complexes coexist, there is increasing statistical likelihood that different proteins (with distinct amino acid sequences) can generate fragments that have similar (or identical) molecular weights. Furthermore, only the fragments containing the amino-and carboxy-termini of a protein are considered during the database searching, limiting the ability to identify the target protein with high confidence, although work is being done to be able to use internal fragments (e.g. fragments resulting from both N- and C-terminal cleavage of the original protein) in a robust way.^{vi,vii}

With knowledge that the use of intact mass alone is insufficient to ascribe a molecular identity when dealing with mixtures of unknowns, native mass spectrometry has developed several strategies to learn about stoichiometry and connectivity of protein complexes.^{viii} But, tandem mass spectrometry and multistage tandem MS (MSⁿ) provides the only means for unambiguous molecular identification of proteins, lipids, ligands, and other small molecules liberated from the intact complex.^{ix} It is therefore imperative to improve the efficiency of fragment ion generation from proteins and complexes. In order to improve the efficiency of top-down fragmentation of intact proteins, one must consider the factors which influence fragmentation such as molecular weight, secondary structure, protein conformation, and charge density.^{x} By nature of native top-down, secondary structure and conformation are preserved immediately prior to fragmentation, and thus the main strategy to improve fragmentation requires consideration of the charge density on the precursor ion. Natively folded proteins bear far fewer charges than their unfolded and denatured counterparts, and therefore becomes a critical variable in top-down fragmentation, as mobile protons are responsible for driving fragmentation in many gas-phase dissociation modalities.^{xi}

There is a need to provide improved tools for native MS studies on target proteins of interest, in particular for applying native MS to mixtures of endogenous proteins and complexes which are present amid the cellular milieu. There is also a need to improve the efficacy of fragment ion generation from proteins and complexes.

### SUMMARY OF INVENTION

The present inventors have developed a peptide mass tag which provides an improved tool for native MS and tandem MS studies. The peptide mass tag advantageously simplifies MS analysis in particular when applying native MS and tandem MS to mixtures of endogenous proteins and complexes. It is able to provide a “diagnostic fragment ion” for unambiguous protein identification of the target protein by native top-down (tandem) mass spectrometry. It also has potential for multiplexing which is of particular utility when low-abundance (e.g., low copy number) proteins are targeted, as distinct specimens (e.g., patient samples) can be pooled without disrupting the initial and unique molecular information ascribed to each sample. The peptide mass tag may be fused to an affinity ligand thereby allowing both for recovery of the target protein and provision of diagnostic fragment ions for a target protein complex.

The experimental results for use of the peptide mass tag illustrate its ability to provide for selective fragmentation allowing unambiguous identification of the target protein and its potential for multiplexing to identify different pools of a target protein and provide information in relation to origin and relative quantitation in pooled samples. The use of the peptide mass tag in combination with affinity ligands with advantageous properties for native MS studies is also shown.

The present invention accordingly provides a peptide mass tag comprising a dipeptide with fragmentation propensity and a binding moiety.

The invention further provides a fusion polypeptide comprising a target polypeptide or an affinity ligand for a target protein fused to a peptide mass tag of the invention.

The invention additionally provides a composition comprising a fusion polypeptide of the invention in complex with a target protein.

The invention also provides a plurality of peptide mass tags of the invention or of fusion polypeptides of the invention, wherein each peptide mass tag is isobaric, optionally wherein each fusion polypeptide is isobaric.

The invention further provides a nucleic acid encoding a peptide mass tag of the invention or a fusion polypeptide of the invention, or a plurality of nucleic acids encoding a plurality of peptide mass tags or fusion polypeptides of the invention. The invention also provides a vector comprising a nucleic acid of the invention, or a plurality of vectors comprising a plurality of nucleic acids of the invention. The invention additionally provides a host cell comprising a nucleic acid or plurality of nucleic acids of the invention, or a vector or plurality of vectors of the invention. The invention further provides a kit or library comprising a plurality of fusion polypeptides of the invention or a plurality of nucleic acids or vectors of the invention.

The invention additionally provides a method of analysing a target protein, the method comprising:
(i) providing
   a sample comprising the target protein, and
   a polypeptide comprising an affinity ligand for the target protein fused to a peptide mass tag, said peptide mass tag comprising a dipeptide with fragmentation propensity;
(ii) exposing the sample to the polypeptide to bind the affinity ligand to the target protein and thus to produce a first composition comprising a polypeptide complex comprising the polypeptide in complex with the target protein;
(iii) exposing the first composition to a capture agent, to reversibly bind the polypeptide complex to the capture agent;
(iv) recovering the polypeptide complex from the capture agent, to provide a second composition comprising the polypeptide complex;
(v) ionising the second composition to produce a tagged gas-phase ion, the tagged gas-phase ion comprising the polypeptide complex; and
(vi) m/z analysing and detecting the tagged gas-phase ion, and/or an ion derived therefrom.

### BRIEF DESCRIPTION OF FIGURES

FIG. 1. Modular domains of the polypeptide tag fused to an affinity reagent. (A) cartoon representation and amino acid sequence of the different domains. (B) Representative native mass spectra (top) and tandem mass spectrometry datasets (middle and bottom) of the affinity reagent in (A). Right shows a detailed view of the polypeptide tag, and the asterisk denotes monoisotopic mass used for mass error calculation (<5 ppm in this case). (C) examples of other sequences of amino acids that are suitable for multiplexed applications, with distinct masses for the diagnostic fragment ions indicated.

FIG. 2. Binding of fusion polypeptide affinity reagent to target protein. (A) native mass spectra of purified hen egg white lysozyme. (B) native mass spectra of the fusion polypeptide affinity reagent. (C) native mass spectra of the fusion polypeptide affinity reagent mixed with lysozyme; peaks for lysozyme disappear, and the new signals that emerge originate from affinity reagent-lysozyme complexes. Only trace quantities of the excess fusion polypeptide affinity reagent are present.

FIG. 3. Isolation of endogenous target protein and non-denaturing elution. (A) workflow depicting the target protein enrichment strategy, which utilizes the fewest number of steps and can be carried out quickly (within minutes). (B) SDS-PAGE analysis of the affinity capture of lysozyme from egg whites using the polypeptide-affinity reagent. Note the clear enrichment of the target protein among a heterogeneous background in elution 1 and elution 2.

FIG. 4. Tandem mass spectrometry analysis of affinity reagent-lysozyme complexes. (A) Low energy native mass spectrum of complexes of affinity reagent and lysozyme. Box with dashed lines corresponds to the isolated peak(B) a spectrum showing the peak that was quadrupole isolated prior to collision induced dissociation. (C) MS² spectrum collected at a low HCD energy, which causes individual components to dissociate via breakage of noncovalent interactions. (D) high energy HCD leads to a MS² spectrum that contains fragment ions of both protein target and affinity reagent complex. Note the peak between m/z 3000 and 4000 is the liberated diagnostic fragment ion that is well separated from all other fragment ions in the m/z spectrum.

FIG. 5 Mixture of three isobaric affinity reagents containing 3 unique fragment ions. The three nanobody-target protein complexes are mixed in approximate equimolar ratio (1:1:1) and because they are isobaric, they are indistinguishable in the native mass spectrum (A) and low energy MS² spectrum (B). High energy MS² results in fragmentation of the protein backbone for both the target protein and the fusion polypeptide affinity reagents; high propensity fragmentation in domain 2 results in the liberation of the diagnostic fragment ions at m/z 3458, 3492, and 3534 Da that are well separated in the m/z dimension from the other sequence ions (C).

FIG. 6. Relative quantitation using raw intensities of diagnostic polypeptide fragment ions. (A) illustrative spectrum showing that the peak intensities of the unique fragment ions appear to be reflective of their relative concentrations. (B) bar graph showing the relative abundance of the different peak intensities extracted after mixing known quantities of fusion polypeptide affinity reagent-lysozyme complexes.

FIG. 7. Identification of wild VGLUT1 protein from a single murine brain using the method of the invention. (A) native mass spectrum of a sample generated after capturing wild-type VGLUT1 from a single murine brain homogenate using an affinity capture reagent comprising an anti-VGLUT1 nanobody connected to a polypeptide tag. Symbols denote the three most abundant, putative charge state distributions with corresponding molecular weights listed as insets. Asterisk denotes the peak that was isolated for tandem MS (B and C). (B) Tandem MS spectrum with labelled fragment ion assignments for the anti-VGLUT1 affinity capture reagent. These fragment ions are overlaid onto the anti-VGLUT1 affinity capture reagent sequence map shown to the right of (B). (C) Tandem MS spectrum of the same peak but searched against the amino acid sequence for wild-type VGLUT1. To the right of the spectrum (C) is the sequence map of wild-type VGLUT1 with overlaid fragment ion matches.

FIG. 8. Demonstration of reproducibility in capturing wild VGLUT1 and capacity to observe co-immunoprecipitating proteins. (A) native mass spectrum of the eluate following capture of VGLUT1 with a nAb specific to it. Two charge state distributions are present, one labelled with black diamonds (a 131,699 kDa protein) and another labelled with nAbs and circles which corresponds to a protein complex weighing 82,904 kDa. The latter is very similar in molecular mass to that anticipated for VGLUT1 bound to the ˜17 kDa nAb. (B) Tandem MS spectrum (MS/MS spectrum) of the 19+ charge state putatively assigned in (A) to VGLUT1-nAb complexes. The fragment ions corresponding to the barcode affixed to the nAb are depicted in grey. The anticipated cleavage at the D/P motif is observed as the inset. The singly charged y-type fragment ions originating from cleavage along aspartic acid repeats are also observed as a series of y₂₁-y₁₈ peaks between m/z 2000-2500. After a database search against mouse VGLUT1, many of the peaks between m/z 1000-2000, and several peaks near m/z 3000-3500 could be assigned to N- and C-terminal fragments. (C) depicts the resulting sequence map of the assigned VGLUT1 fragment ions. (D) Tandem MS spectrum (MS/MS spectrum) of the 23+ charge state assigned to the copurifying protein (black diamonds in (A)). polypeptide tag on the nanobody (affinity capture reagent). Several of the fragment ions were matched to GrpE1 (˜23 kDa protein), following an open database search against the mouse proteome. (E) Sequence map of GrpEl showing the position of the assigned fragment ions. To account for the intact mass of ˜132 kDa, GrpE1 is anticipated to be a part of a larger protein complex that copurifies with VGLUT1 but does not bind to the nAb.

### BRIEF DESCRIPTION OF SEQUENCES

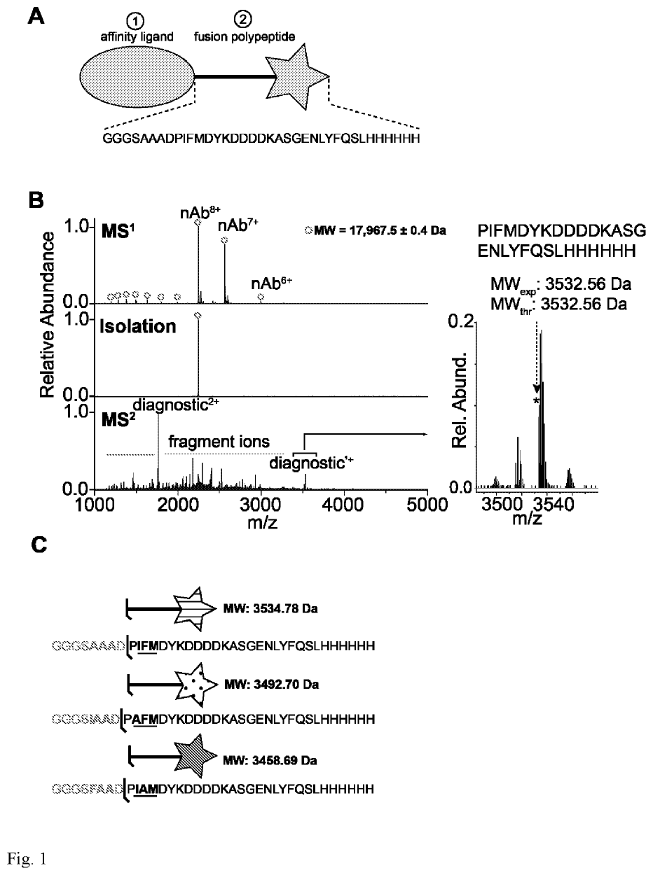

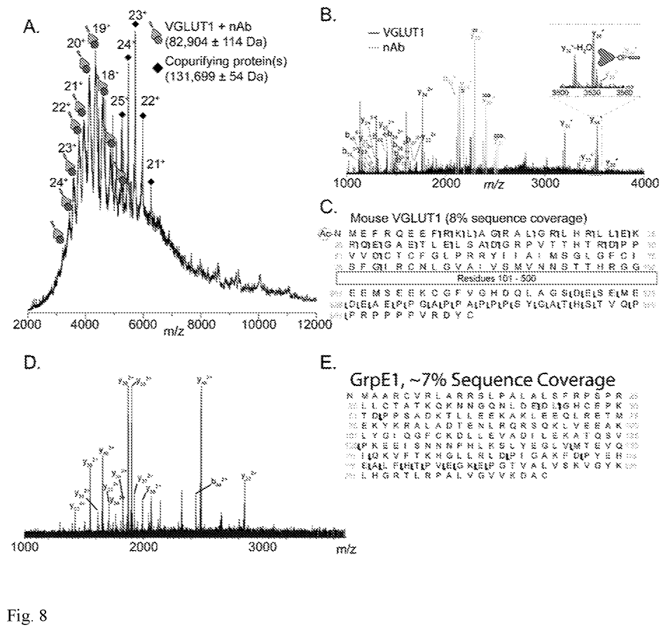

SEQ ID NOs: 1-3=full length sequences in FIGS. 1A and 1C
SEQ ID NOs: 4-6=N-terminal sequences of FIG. 1C
SEQ ID NOs: 7-9=C-terminal sequences of FIG. 1C
SEQ ID NO: 10=FLAG peptide epitope
SEQ ID NO: 11=c-myc peptide epitope
SEQ ID NO: 12=Rho-1D4 peptide epitope
SEQ ID NO: 13=HA peptide epitope
SEQ ID NOs 14-18=flexible linkers
SEQ ID NO: 19=Anti-VGLUT1 fusion reagent
SEQ ID NO: 20=VGLUT1 (mouse)
SEQ ID NO: 21=cleavage sequence for tobacco etch protease
SEQ ID NO: 22=cleavage sequence for rhinoviral 3C protease
SEQ ID NOs: 23-24=sequences disclosed in Example 1
SEQ ID NOs 25-27=sequences disclosed in FIG. 8

### DETAILED DESCRIPTION

It is to be understood that different applications of the disclosed products and methods may be tailored to the specific needs in the art. It is also to be understood that the terminology used herein is for the purpose of describing particular embodiments of the invention only, and is not intended to be limiting. In addition as used in this specification and the appended claims, the singular forms “a”, “an”, and “the” include plural referents unless the content clearly dictates otherwise. All publications, patents and patent applications cited herein, whether supra or infra, are hereby incorporated by reference in their entirety.

### Peptide Mass Tag

The invention provides a peptide mass tag which is able to act as a reporter for a target protein/target protein complex. The peptide mass tag may be provided unfused to any other polypeptide, for example in the context of a nucleic acid or vector which may be used for cloning an encoding nucleic acid sequence in frame with the peptide mass tag, as discussed below. Alternatively, the peptide mass tag may be fused to a polypeptide, such as a target polypeptide or an affinity ligand for a target protein.

The peptide mass tag comprises a dipeptide with fragmentation propensity and a binding moiety and may optionally further comprise a proton-donating motif. The peptide mass tag may further be described as comprising a reporter portion which comprises one amino acid of the dipeptide with fragmentation propensity and the distal portion of the peptide mass tag when fused to a polypeptide of interest. The peptide mass tag typically has a mass of 1 kDa to 8 kDa. The peptide mass tag is typically at least 10 amino acids in length. The peptide mass tag may have a length ranging from 10 to 80 amino acids. The peptide mass tag is preferably less than 40 amino acids in length. The mass of the reporter portion of the peptide mass tag is generally less than 8 kDa and preferably less than 6 kDa. For instance it is typically from 2 kDa to 8 kDa and preferably is from 3 kDa to 5 kDa. This corresponds to a region of the mass spectrum that is typically sparsely populated by product ions in tandem MS experiments and thus suitable for providing a unique reporter ion.

The peptide mass tag may comprise one or more linkers between the dipeptide with fragmentation propensity, the binding moiety, and the proton-donating motif (where present). Suitable linkers are as discussed below in connection with fusion polypeptides.

### Dipeptide with Fragmentation Propensity

The dipeptide with fragmentation propensity promotes fragmentation of the peptide mass tag during MS, typically tandem MS. The fragmentation is typically in the gas phase. The fragmentation typically breaks the peptide backbone of the mass tag at the dipeptide with fragmentation propensity. The fragmentation accordingly breaks the peptide bond between the two amino acids of the dipeptide. The fragmentation thus results in at least two fragment ions comprising sequence derived from the peptide mass tag, each comprising one amino acid of the dipeptide. The skilled person assisted by the teaching of the application and their common general knowledge is able to select dipeptides with suitable characteristics providing for fragmentation propensity. The ability of selected dipeptides to provide adequate fragmentation for MS analysis may be evaluated empirically by the skilled person with reference to the fragmentation efficiency illustrated in the examples. The fragmentation efficiency may be defined as the % total of fragment ion abundance relative to the energy required to produce the fragment, and may be as described in Wysocki et al (J. Mass Spectrom. 2000, 35, 1399-1406) infra, incorporated by reference herein. Suitable characteristics for the dipeptide include low peptide bond strength. Dipeptides may be selected according to the teaching of Haverland et al infra^{xiii}, incorporated by reference herein. Examples of suitable dipeptides include D/E-X, I/L/V-X, X-P or F-W, wherein X is any amino acid and “/” between amino acids indicates that these are alternatives. Examples of D-X sites are D-Y, D-G and D-C. A preferred dipeptide is D/E/I/L/V-P, which may be DP, EP, IP, LP, or VP. A particularly preferred dipeptide is DP.

The dipeptide may be positioned anywhere within the peptide mass tag, provided that its fragmentation generates a suitable reporter ion. The dipeptide is typically positioned sufficiently proximal to the relevant terminus of the tag for fusion to a polypeptide of interest such that it generates a suitable reporter ion. Where a plurality of peptide mass tags is used in multiplex applications, the dipeptide of each peptide mass tag is positioned within the isobaric multiplet of amino acids of each peptide mass tag, as discussed below. When a proton-donating motif is present in the peptide mass tag, the dipeptide is typically positioned sufficiently proximal to the motif to allow for proton donation to assist backbone cleavage of the dipeptide.

### Binding Moiety

The binding moiety permits recovery of the target protein/complex or of a complex between the affinity ligand and target protein when the peptide mass tag is fused to an affinity ligand. Any suitable binding moiety may be used provided the target protein/complex is able to be eluted from a capture agent for the binding moiety under non-denaturing conditions. The binding moiety thus allows for purification and elution of the target protein/complex without substantial denaturation of the target protein/complex. Advantageously, this allows for preservation of non-covalent interactions between the target protein and other molecules with which it may be in complex, such that native MS analysis is possible. The skilled person is aware of suitable binding pairs (binding moiety and capture agent) which allow for purification of a target complex and subsequent elution without disruption of intermolecular interactions. The binding interaction between the binding moiety and the capture agent is thus typically reversible under non-denaturing conditions. The binding interaction may be reversible without substantially interfering with intermolecular interactions within the target complex. Suitable binding pairs of a binding moiety and a capture agent may for example be a ligand and receptor, an epitope and an anti-epitope antibody, such as a peptide and anti-peptide antibody, or a pair of chelating agents. The binding interaction may be reversible by competitive displacement of the binding moiety of the peptide mass tag with free ligand/free peptide/a stripping agent.

The capture agent typically has high affinity for the binding moiety, such that sufficient quantities of the target protein/complex may be recovered for MS analysis. The capture agent may be immobilised on a solid support, such as a resin or beads, to assist recovery of the target protein/complex by its immobilisation on the solid support through interaction of the binding moiety and the capture agent. Suitable capture agents are discussed in more detail below.

Typically the binding moiety is of low molecular weight to assist reduction in size of the peptide mass tag. The binding moiety may be of less than 3000, more preferably less than 2000, or less than 1500 Da in mass. Use of a low molecular weight binding moiety also advantageously means that corresponding free ligand/free peptide used for competitive displacement of the binding moiety may be easily removed from a sample prior to MS analysis, for example by dialysis or filtration. The binding moiety may be a low molecular weight compound or may be a peptide epitope, such as a linear peptide epitope. The binding moiety may be a chelating agent for a metal ion, such as a polyhistidine sequence (His tag) described herein which may be used to chelate Ni-ions. In this aspect, the capture agent may be an Ni-NTA resin which binds to the polyhistidine sequence. A low molecular weight compound may be any such compound for which a specific capture agent may be provided. As an example, the binding moiety may be biotin and the capture agent streptavidin. The peptide mass tag may thus be biotinylated, optionally at a specific site. A peptide epitope is typically a short peptide epitope. A short peptide epitope may be of 5-15, more preferably 5-10 amino acids in length. Suitable short peptide epitopes include FLAG, c-Myc, Rho-1D4 and HA. Exemplary sequences for short FLAG, c-Myc, Rho-1D4 and HA are provided respectively as SEQ ID NOs: 10-13. Other suitable binding moieties include AviTag, calmodulin-tag, polyglutamate tag, E-tag, S-tag, SBP-tag, Softag 1, Softag 3, Strep-tag, TC tag, V5 tag, VSV-tag, Xpress tag, Isopeptag, SpyTag, SnoopTag, BCCP (Biotin Carboxyl Carrier Protein), Glutathione-S-transferase-tag, Green fluorescent protein-tag, Halo-tag, Maltose binding protein-tag, Nus-tag, Thioredoxin-tag, Strep-tag, Skin permeating and cell entering (SPACE)-tag, TD1-tag, magainin tag, TAT-tag, penetratin-tag, cell penetrating peptide (CPP)-tag, and Fc tag.

The binding moiety is typically covalently attached to the peptide mass tag. The binding moiety may be covalently attached to a side-group of the peptide backbone. Alternatively, the binding moiety may be a peptide epitope forming part of the amino acid sequence of the peptide mass tag, as discussed above.

The binding moiety may be located anywhere within the peptide mass tag. The binding moiety is suitably provided at a location accessible to binding by the capture agent when fused to a polypeptide of interest. The skilled person is able to determine a suitable location within the peptide mass tag for inclusion of the binding moiety.

### Proton-Donating Motif

The peptide mass tag may optionally comprise a proton-donating motif. A proton-donating motif may be provided to enhance fragmentation of the peptide mass tag at the dipeptide with fragmentation propensity, where desirable. However, it is considered that adequate fragmentation will be obtained in many cases without inclusion of a proton-donating motif. A proton-donating motif may be used where a given peptide mass tag or polypeptide is observed to have lower fragmentation propensity. Proton donation or adduction from the proton-donating motif toward the dipeptide may enhance cleavage of the peptide backbone at the dipeptide. The proton-donating motif may enhance generation of the desired reporter fragment ions from the peptide mass tag, including 1+ and/or 2+ charge states.

The proton-donating motif typically contains one or more basic amino acids and may also be referred to herein as a polybasic site. The basic amino acids may be histidine, lysine and/or arginine residues. The polybasic site may comprise at least two, at least three, at least four, at least five or at least six basic acids, such as at least two, at least three, at least four, at least five or at least six histidine, lysine or arginine residues. A preferred example of a polybasic site is a polyhistidine site. A hexahistidine site is particularly preferred. A polyhistidine site, such as a hexahistidine site may also be employed as the binding moiety for a capture agent, as discussed above. In this aspect, the polyhistidine site may act both as the proton-donating motif and the binding moiety of the peptide mass tag. A hexahistidine site may further advantageously allow for purification of a polypeptide comprising the peptide mass tag by Ni-NTA chromatography. In particular, a hexahistidine site may be used to purify an affinity ligand comprising the peptide mass tag from a host cell used for recombinant expression, such as a prokaryotic host cell.

### Multiplexing

The peptide mass tags of the invention are particularly suitable for use in multiplex applications, for example where different pools/samples of a target protein/complex are to be analysed in combination. In such applications, a plurality of different peptide mass tags may be used, each one to be associated with a specific pool/sample of the target protein/complex. The particular properties of the target protein/complex in each pool/sample may then be determined by reference to the particular peptide mass tag used for the relevant pool/sample. For example, a relative quantitation of the target protein/complex in each pool/sample may be made based on the abundance of each particular peptide mass tag.

A plurality of peptide mass tags may thus be provided. Each peptide mass tag of the plurality is typically isobaric, i.e. has the same mass/molecular weight. This advantageously allows for detection of the intact tag in each sample/pool based on the same m/z and for a common baseline against which variation in amino acid sequence may be used to provide for a difference in mass of reporter ions. Each peptide mass tag further typically has the same amino acid composition and typically the same dipeptide having fragmentation propensity, the same binding moiety and the same proton-donating motif (when present). However, each peptide mass tag typically differs in that an isobaric multiplet of amino acids comprising the dipeptide varies in sequence in each peptide mass tag. Thus, the same amino acids are present in the isobaric multiplet of amino acids but the order i.e. sequence of those amino acids differs in each tag. Each isobaric multiplet of amino acids thus has a unique sequence of amino acids. The dipeptide is typically present at the same position in each tag.

The differing sequence of each isobaric multiplet of amino acids results in a reporter ion (also referred to as a reporter portion or reporter fragment ion) of a different mass being generated on cleavage of the dipeptide of each peptide mass tag. Accordingly, it is possible to distinguish the origin of each target protein/complex (for example the pool or sample from which it is derived) based on the unique mass of the reporter fragment ion associated with the peptide mass tag of origin, e.g. the peptide mass tag used in a particular pool or sample.

Advantageously, the use of a varying isobaric multiplet of amino acids in each peptide mass tag as described above avoids the need for differing chemical/isotopic labelling of each tag as used in other tagging approaches. However, additionally or alternatively, variation may be provided between each peptide mass tag of the plurality by inclusion of a reactive amino acid as part of a multiplet of amino acids comprising the dipeptide. The reactive amino acid may then be controlled to be protected or available for chemical conjugation and/or conjugated to different chemical partners (of differing mass) in each peptide mass tag of the plurality, again resulting in a reporter fragment of a different mass being generated from each peptide mass tag on cleavage of the dipeptide. The reactive amino acid may be a cysteine. Additionally or alternatively, variation may further be provided between each peptide mass tag of the plurality by distinct isotopic labelling of peptide mass tags of the plurality.

The isobaric multiplet may comprise the dipeptide and only one additional amino acid. That additional amino acid may or may not be positioned in the reporter portion of the mass tag. Thus, at least two permutations of the peptide mass tag having different masses of reporter ion are possible; one containing the additional amino acid and one without. Moreover, where the two amino acids of the dipeptide each have a differing mass, the mass of the reporter portion will vary dependent on which of those two amino acids are present in the reporter portion and consequently four different masses of the reporter portion are possible. Such aspects, wherein the number of amino acids in the isobaric multiplet on either of the dipeptide may differ, are described in more detail in the “multiplexing methods” section below.

Typically, the isobaric multiplet comprises the same number of amino acids on either side of the dipeptide. The isobaric multiplet of amino acids typically comprises the dipeptide and at least one amino acid on either side of the dipeptide, i.e. comprises the dipeptide and two additional amino acids. In this aspect, the amino acid on either side of the dipeptide may be switched in two different peptide mass tags, resulting in a reporter fragment ion of a different mass being generated from each tag, based on the differing amino acid present in the reporter portion of each tag. Two different pools/samples of a target protein/complex may thus be differentiated by using one of the two different peptide mass tags in each pool/sample. More preferably, the isobaric multiplet comprises the dipeptide and at least two or at least three additional amino acids on each side of the dipeptide, to increase multiplexing potential. The isobaric multiplet may comprise the dipeptide and between three and five additional amino acids on each side of the dipeptide. This range provides for multiplexing of a large number of pools/samples, as discussed below, while advantageously reducing overall size of the peptide mass tag. In some aspects, the isobaric multiplet may comprise six, seven or eight additional amino acids on each side of the dipeptide to further increase multiplexing potential.

The additional amino acids on either side of the dipeptide must vary in sequence in each peptide mass tag at at least one position. Additionally, the at least one varying amino acid is typically of a different mass. Thus, pairs of amino acids having identical molecular weights (I/L; K/Q) are typically not used as a basis for at least one variation between peptide mass tags. Additionally, the amino acids D and P may be avoided as additional amino acids in the multiplet based on their preferred use in the dipeptide with fragmentation propensity. As an illustration of the huge multiplexing potential available based on sequence variation of the multiplet in each peptide mass tag, multiplet sequences based on the sixteen natural amino acids other than I,/L, K/Q, D and P would allow for eight additional amino acids on each side of the dipeptide, yielding 12,870 possible different sequence permutations. In such aspects, the possible combinations for each multiplet, factoring in redundancies, may be determined based on the following formula:

$${C}_{( n , r )} = \frac{n !}{r ! \times ( n - r ) !}$$

in which C=the number of combinations, n=the total number of residues in the multiplet, and r=the reduced set, or fixed number of elements that can change. Applying this formula to example numbers for total additional amino acid residues on each side of the dipeptide in the multiplet provides the following combinations: 1 amino acid: C(2,1)=2 combinations; 2 amino acids: C(4,2)=6 combinations; 3 amino acids: C(6,3)=20 combinations; 4 amino acids: C(8,4)=70 combinations; 5 amino acids: C(10,5)=252 combinations; 8 amino acids: C(16,8)=12,870 combinations. Accordingly, extensive variation of the multiplet is possible solely based on sequence permutations based on the naturally occurring amino acids. Variation of the multiplet may additionally or alternatively be provided by incorporation of unnatural or covalently modified amino acids in the sequence.

The isobaric multiplet of amino acids comprising the dipeptide is generally of at least four amino acids in length. Typically, the isobaric multiplet is at least five, at least six, at least seven, preferably at least eight amino acids in length. The isobaric multiplet may be between four and eighteen amino acids in length, between four and sixteen amino acids in length, between eight and eighteen amino acids in length, or between eight and sixteen amino acids in length.

In aspects relating to multiplexing, the invention thus provides a plurality of peptide mass tags, wherein each peptide mass tag is isobaric as described above. Each of the peptide mass tags of the plurality thus comprises an isobaric multiplet of amino acids comprising the dipeptide with fragmentation propensity, wherein the multiplet varies in sequence in each peptide mass tag. The isobaric peptide mass tags of the plurality, each varying in sequence, may be described as variant peptide mass tags. The plurality of peptide mass tags comprises at least two variant peptide mass tags, and may comprise at least three, at least four, at least five, at least six, at least eight, at least ten, at least twelve, at least sixteen, at least twenty or at least thirty peptide mass tags. The plurality of peptide mass tags may comprise up to 20,000, up to 10,000, up to 1000, up to 500, up to 200 or up to 100 variant peptide mass tags. The plurality of peptide mass tags may comprise between 2 and 500, 2 and 300, 6 and 300, 10 and 300, 20 and 300, 40 and 300, 70 and 300 variant peptide mass tags.

### Fusion Polypeptide

The invention further relates to a polypeptide comprising a target polypeptide or an affinity ligand for a target polypeptide, fused to a peptide mass tag of the invention. The said polypeptide thus always comprises a peptide mass tag of the invention. Such a polypeptide may also be described as a fusion polypeptide. The target polypeptide or affinity ligand may be described as the fusion partner.

The peptide mass tag may be fused to the target polypeptide where it is desired to express such a fusion polypeptide recombinantly, e.g. in a model system to study interactions of the target polypeptide. The target polypeptide may be any polypeptide of interest, such as any mammalian, preferably human polypeptide of interest. In some embodiments, a fusion polypeptide of a target polypeptide and peptide mass tag may not comprise a binding moiety as part of the peptide mass tag, i.e. the peptide mass tag may solely comprise the dipeptide with fragmentation propensity (optionally comprised in an isobaric multiplet of amino acids), and optionally a proton-donating motif. In such embodiments, a ligand (such as a native ligand) for the target polypeptide (e.g. immobilised on a solid support) may be used to isolate the target polypeptide/complex, by binding of the target polypeptide.

Alternatively, the peptide mass tag may be fused to an affinity ligand for the target polypeptide. In this aspect, the affinity ligand may be used to recover the native target polypeptide/complex in any context, e.g. from endogenous tissue or from a patient sample, without any modification of the target polypeptide.

The fusion polypeptide may comprise the peptide mass tag at any location. The peptide mass tag is suitably fused at a location which is accessible for binding of the binding moiety by the capture agent. The fusion location is suitably also one which does not disrupt one or more native binding interactions of the target polypeptide/complex, or does not substantially disrupt binding of the affinity ligand to the target polypeptide. The N- or C-terminus of the peptide mass tag may preferably be fused to a terminus of the fusion partner. The fusion may be to the N- or C-terminus of the fusion partner.

The fusion is typically a covalent linkage. The fusion may be direct or may be indirect, such as by a linker, as discussed below. The fusion may be a genetic fusion. Typically, the fusion polypeptide is a genetic fusion if the whole construct is expressed from a single polynucleotide sequence. The coding sequences of the peptide mass tag of the invention and of the fusion partner may be combined in any way to form a single polynucleotide sequence encoding the construct. They may be genetically fused in any configuration.

Where the peptide mass tag is attached to the fusion partner using one or more linkers, any suitable linkers may be used. Suitable linkers include, but are not limited to, chemical crosslinkers and peptide linkers. Peptide linker are preferred if the peptide mass tag and fusion partner are genetically fused. Preferred linkers are amino acid sequences (i.e. peptide linkers). The length, flexibility and hydrophilicity of the peptide linker are typically designed such that it does not to disturb the functions of the polypeptide of the invention. Preferred flexible peptide linkers are stretches of 2 to 20, such as 3, 4, 6, 8, 10 or 16, serine and/or glycine amino acids. More preferred flexible linkers include (SG)1, (SG)2 (SEQ ID NO:14), (SG)3(SEQ ID NO:15), (SG)4(SEQ ID NO:16), (SG)5 (SEQ ID NO:17) and (SG)8 (SEQ ID NO:18) wherein S is serine and G is glycine. Suitable linkers may additionally or alternatively comprise amino acid sequences that can be cleaved by proteases, for example, tobacco etch protease ENLYFQ/(SEQ ID NO: 21) and rhinoviral 3C protease (LEVLFQ/GP)(SEQ ID NO: 22).

The invention also provides a plurality of fusion polypeptides as described above, wherein the peptide mass tag of each fusion polypeptide is isobaric, optionally wherein each fusion polypeptide is isobaric. Typically, the peptide mass tag of each fusion polypeptide comprises an isobaric multiplet of amino acids comprising the dipeptide with fragmentation propensity, wherein the multiplet varies in sequence in each peptide mass tag. Where the fusion is to a target polypeptide, each fusion polypeptide may comprise (i) a target-specific portion comprising the target polypeptide, the amino acid of the dipeptide with fragmentation propensity proximal to the target polypeptide, and any intervening amino acid(s); and (ii) a reporter portion comprising the other amino acid of the dipeptide with fragmentation propensity and the remainder of the peptide mass tag. Where the fusion is to an affinity ligand, each fusion polypeptide may comprise (i) a target-binding portion comprising the affinity ligand, the amino acid of the dipeptide with fragmentation propensity proximal to the affinity ligand, and any intervening amino acid(s); and (ii) a reporter portion comprising the other amino acid of the dipeptide with fragmentation propensity and the remainder of the peptide mass tag. Preferred characteristics such as masses for the reporter portion are as discussed above, with the mass being typically less than 6 kDa, and preferably from 3 kDa to 5 kDa.

### Affinity Ligand

An affinity ligand is any form of ligand capable of specifically binding to a target polypeptide of interest, with sufficient affinity to allow for recovery of a complex comprising the target polypeptide and the affinity ligand. An affinity ligand may for example be a peptide ligand, a nucleic acid aptamer or an antigen-binding molecule, such as an antibody or antibody mimetic. A peptide ligand is any peptide or peptidomimetic having specificity for a target polypeptide of interest e.g. that represents a portion of a native ligand for the target polypeptide or a derivative thereof. An antibody as referred to herein may relate to whole antibodies (i.e. comprising the elements of two heavy chains and two light chains inter-connected by disulphide bonds) as well as antigen-binding fragments thereof. An antibody typically comprises an immunologically active portion of an immunoglobulin (Ig) molecule, i.e. that contains an antigen binding site that specifically binds (immunoreacts) with an antigen. Each heavy chain is comprised of a heavy chain variable region (abbreviated herein as HCVR or VH) and at least one heavy chain constant region. Each light chain is comprised of a light chain variable region (abbreviated herein as LCVR or VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR).

The antibody may be a monoclonal antibody. Monoclonal antibodies (mAbs) of the invention may be produced by a variety of techniques, including conventional monoclonal antibody methodology, for example those disclosed in “Monoclonal Antibodies: a manual of techniques” (Zola H, 1987, CRC Press) and in “Monoclonal Hybridoma Antibodies: techniques and applications” (Hurrell J G R, 1982 CRC Press). A monoclonal antibody may be chimeric, CDR-grafted, human or humanised. The antibody of the invention may be multispecific, such as bispecific. A bispecific antibody of the invention binds two different epitopes, which may be in the same protein or in different proteins. Methods to prepare multispecific, e.g. bispecific, antibodies are well known in the art. The antibody may be a polyclonal antibody. A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

Preferably, affinity ligands of relatively small size, preferably below 20 kDa in size, are used according to the invention, in order to reduce spectral complexity. Preferably, a fusion polypeptide comprising the affinity ligand and the peptide mass tag is below 30 kDa in size, such as between 10 kDa and 25 kDa in size. Accordingly, the antibody is preferably an antigen-binding fragment or antibody derivative. An antigen-binding fragment typically comprises the complementarity-determining regions (CDRs) that interact with the antigen, such as one, two, three, four, five or six CDRs. In some embodiments, the antigen-binding fragment further comprises framework regions surrounding the CDRs. Preferably the antigen-binding fragment or antibody derivative does not include a constant region. The antigen-binding fragment or derivative may be a single chain antibody, a single chain variable fragment (scFv), a variable fragment (Fv), a fragment antigen-binding region (Fab), an Fab′, an F(ab′)₂, a single-domain antibody (sdAb), a VHH antibody, a nanobody (a camelid-derived single-domain antibody), a shark IgNAR-derived single-domain antibody fragment (VNAR), a sybody, a diabody, a triabody, or an active component of fragment of any thereof. A preferred example of a single domain antibody is a sybody. Sybodies are described for example in publications by Zimmerman et al (xxii-xxiii). A particularly preferred antibody is a VHH antibody or a nanobody. A nanobody derived from a camelid, shark or other nanobody-producing animal is especially preferred. Additionally antibody mimetics, in particular mimetics of reduced size, such as anticalins, may be used.

It is further preferred that an affinity ligand, such as an antibody, does not include extensive post-translational modifications, such as glycosylation, that may increase spectral complexity. The affinity ligand may be produced in a prokaryotic expression system to minimise or avoid post-translational modification. The affinity ligand may alternatively be designed without any or with a decreased number of glycosylation sites (such as N-linked glycosylation sites, for example sequons (NXS/T) to allow for eukaryotic expression without/with reduced glycosylation.

An affinity ligand “specifically binds” to a polypeptide when it binds with preferential or high affinity to that polypeptide but does not substantially bind, does not bind or binds with only low affinity to other polypeptides. Where a target polypeptide is abundant, it may not be necessary to recover substantially all of the target polypeptide for MS analysis and thus the particular affinity required for the affinity ligand may be selected by the skilled person according to the application. An affinity ligand binds with preferential or high affinity if it binds with a K_{D} of 1×10-7 M or less, more preferably 5×10-8 M or less, more preferably 1×10-8 M or less or more preferably 5×10-9 M, 4×10-9 M, 3×10-9 M or less. It is particularly preferred that the K_{D} is 2×10-9 M or less or 1×10-9 M or less. An affinity ligand binds with low affinity if it binds with a K_{D} of 1×10-6 M or more, more preferably 1×10-5 M or more, more preferably 1×10-4 M or more, more preferably 1×10-3 M or more, even more preferably 1×10-2 M or more. A variety of protocols for competitive binding or immunoradiometric assays to determine the specific binding capability of compounds, such as antibodies or antibody constructs are well known in the art (see for example Maddox et al, J. Exp. Med. 158, 1211-1226, 1993). The KD value can be measured, for example, by ELISA or Surface Plasmon Resonance (Biacore) at 25° C. Binding affinity (K_{D}) may be quantified by determining the dissociation constant (Kₐ) and association constant (Kₐ) for an antibody and its target. For example, the antibody may have an association constant (Kₐ) of ≥10000 M⁻¹s⁻¹, ≥50000 M⁻¹s⁻¹, ≥100000 M⁻¹s⁻¹, ≥200000 M⁻¹s⁻¹ or ≥500000 M⁻¹s⁻¹, and/or a dissociation constant (K_{d}) of ≤0.001 s⁻¹, ≤0.0005 s⁻¹, ≤0.004 s⁻¹, ≤0.003 s⁻¹, ≤0.002 s⁻¹ or ≤0.0001 s⁻¹.

The affinity ligand typically binds to the target protein with high efficiency. This enables the polypeptide ligand to recover most, if not all, of the target protein from a sample. The affinity ligand may bind to the target protein with an efficiency of at least 50%, such as at least 60%, 70%, 80%, preferably at least 90%, more preferably at least 95%. By this is meant that, where a fusion polypeptide comprising the affinity ligand is mixed with the target protein in equimolar quantities, typically at least 50% or preferably at least 90% of the fusion polypeptide will bind to the target protein. A high efficiency of binding between target protein and affinity ligand is particularly important where the target protein is scarce in the sample. If the target protein is highly abundant in the sample, a relatively low efficiency of binding between affinity ligand and target protein may be acceptable.

### Complex

The invention further provides a complex comprising a target polypeptide and a fusion polypeptide of the invention comprising an affinity ligand for the target polypeptide fused to a peptide mass tag. The affinity ligand and target polypeptide are bound together in the complex. The complex is typically comprised in a composition. The composition may be a cell or tissue lysate or other sample described herein.

### Nucleic Acids, Vectors, Host Cells and Organisms

The invention further relates to a nucleic acid encoding a peptide mass tag or fusion polypeptide of the invention. The invention further relates to a plurality of such nucleic acids or vectors, wherein the peptide mass tag and/or a fusion polypeptide comprising the peptide mass tag is isobaric, as described above. A nucleic acid is a polymer comprising two or more nucleotides. The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate.

The nucleotides in the polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The nucleic acid is typically a deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The nucleic acid may be single stranded or double stranded.

The polynucleotide sequence of the nucleic acid encodes the peptide mass tag or fusion polypeptide on the basis of the genetic code, including its degeneracy.

Polynucleotide sequences may be derived and replicated using standard methods in the art, for example using PCR involving specific primers. It is straightforward to generate polynucleotide sequences using such standard techniques.

The invention also relates a vector comprising a nucleic acid of the invention i.e. comprising a polynucleotide sequence encoding for the peptide mass tag or fusion polypeptide. The vector may be a cloning vector. An amplified sequence may be incorporated into a recombinant replicable vector such as a cloning vector. The sequence of the target polypeptide or of the affinity ligand may be cloned in frame with a polynucleotide sequence encoding the peptide mass tag present in an existing vector (also described as an empty cloning vector). The vector may be used to replicate the polynucleotide in a compatible host cell. Thus polynucleotide sequences may be made by introducing the polynucleotide into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The vector may be an expression vector. The polynucleotide sequence of the peptide mass tag or of the fusion polypeptide may be cloned into any suitable expression vector. The sequence of the target polypeptide or of the affinity ligand may be cloned in frame with a polynucleotide sequence encoding the peptide mass tag present in an existing vector (also described as an empty expression vector). In an expression vector, the polynucleotide of the invention is typically operably linked to a control sequence which is capable of providing for the expression of the polynucleotide by the host cell. Such expression vectors can be used to express fusion polypeptides of the invention.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The term “control sequence” is intended to include promoters, enhancers, internal ribosomal entry sites (IRES), and other expression control elements (e.g. transcription termination signals, such as polyadenylation signals and poly-U sequences). Such control sequences are described, for example, in Goeddel, GENE EXPRESSION TECHNOLOGY: METHODS IN ENZYMOLOGY 185, Academic Press, San Diego, Calif. (1990).

Control sequences may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “control sequence” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. With regards to control sequences, mention is made of U.S. patent application Ser. No. 10/491,026. With regards to promoters, mention is made of PCT publication WO 2011/028929 and U.S. application Ser. No. 12/511,940.

The expression vector may then be introduced into a suitable host cell. Thus, a fusion polypeptide of the invention can be produced by inserting an encoding polynucleotide sequence into an expression vector, introducing the vector into a compatible bacterial host cell, and growing the host cell under conditions which bring about expression of the polynucleotide or combination. The vectors may be for example, plasmid, virus or phage vectors provided with an origin of replication, optionally a promoter for the expression of the said polynucleotide and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The vector may be used to administer a nucleic acid of the invention to a subject. Conventional viral and non-viral based gene transfer methods can be used to introduce the polynucleotide into cells. Non-viral vector delivery systems include DNA plasmids, RNA, naked nucleic acid, and nucleic acid complexed with a delivery vehicle, such as a liposome. Methods of non-viral delivery of nucleic acids include lipofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. The preparation of lipid: nucleic acid complexes, including targeted liposomes such as immunolipid complexes, is well known to one of skill in the art (sec, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Conventional viral based expression systems could include retroviral, lentivirus, adenoviral, adeno-associated (AAV) and herpes simplex virus (HSV) vectors for gene transfer. Methods for producing and purifying such vectors are known in the art.

The vector may be delivered using nanoparticle delivery systems. Such delivery systems include, but are not limited to, lipid-based systems, liposomes, micelles, microvesicles, exosomes, and gene gun. With regard to nanoparticles that can deliver RNA, sec, e.g., Alabi et al., Proc Natl Acad Sci U S A. 2013 Aug. 6;110 (32):12881-6 Zhang et al., Adv Mater. 2013 Sep. 6;25(33):4641-5; Jiang et al., Nano Lett. 2013 Mar. 13;13(3):1059-64; Karagiannis et al., ACS Nano. 2012 Oct. 23;6(10):8484-7; Whitehead et al., ACS Nano. 2012 Aug. 28;6(8):6922-9 and Lee et al., Nat Nanotechnol. 2012 Jun. 3;7(6):389-93. Lipid Nanoparticles, Spherical Nucleic Acid (SNA™) constructs, nanoplexes and other nanoparticles (particularly gold nanoparticles) are also contemplated as a means for delivery of a polynucleotide of the invention.

The invention also provides a host cell which comprises a nucleic acid of the invention (polynucleotide) or a vector of the invention. The host cell may be used to replicate the polynucleotide or vector. The host cell may be used to express a fusion polypeptide of the invention in vitro.

Host cells will be chosen to be compatible with the cloning or expression vector used to transform the cell. Suitable conditions are known in the art (see, for instance, Sambrook, J. and Russell, D. supra).

Suitable cells for use in the invention include prokaryotic cells and eukaryotic cells. The prokaryotic cell is preferably a bacterial cell. Suitable bacterial cells include, but are not limited to, Escherichia coli, Corynebacterium and Pseudomonas fluorescens. Any E. coli cell with a DE3 lysogen, for example C41 (DE3), BL21 (DE3), JM109 (DE3), B834 (DE3), TUNER, Origami and Origami B, can express a vector comprising the T7 promoter.

Suitable eukaryotic cells include, but are not limited to, Saccharomyces cerevisiae, Pichia pastoris, filamentous fungi, such as Aspergillus, Trichoderma and Myceliophthora thermophila C1, baculovirus-infected insect cells, such as Sf9, Sf21 and High Five strains, non-lytic insect cells, Leishmania cells, plant cells, such as tobacco plant cells, and mammalian cells, such as Bos primigenius cells (Bovine), Mus musculus cells (Mouse), Chinese Hamster Ovary (CHO) cells, Human Embryonic Kidney (HEK) cells, Baby Hamster Kidney (BHK) cells and HeLa cells. Other preferred mammalian cells include, but are not limited to, PC12, HEK293, HEK293A, HEK293T, CHO, BHK-21, HeLa, ARPE-19, RAW264.7 and COS cells.

The cell may be isolated, substantially isolated, purified or substantially purified. The cell is isolated or purified if it is completely free of any other components, such as culture medium or other cell types. The cell is substantially isolated if it is mixed with carriers or diluents, such as culture medium and others discussed above and below, which will not interfere with its intended use. Alternatively, the host cell of the invention may be present in a growth matrix or immobilized on a surface as discussed below.

The invention also provides a non-human organism transformed with a nucleic acid or vector of the invention, such as by any technique described above. The non-human organism may be a mammal, such as a rodent. The rodent may be a mouse or rat. The non-human organism may be described as a model organism. Typically, the nucleic acid or vector encodes a fusion polypeptide comprising a target polypeptide fused to a peptide mass tag. The fusion polypeptide may thus be expressed in a non-human organism and its interactions then studied by MS using the peptide mass tag, after isolation of the target polypeptide/complex.

### Kit and Library

The invention further relates to a kit or library comprising a plurality of fusion polypeptides, nucleic acids or vectors as described above, wherein the peptide mass tag and/or fusion polypeptide comprising the peptide mass tag is isobaric, as described above. The kit may comprise each member of the plurality of agents spatially separated from one another, for example in different containers. The kit may comprise details of the mass of the reporter portion of each peptide mass tag. The kit may further comprise instructions for use in any method described herein. The library may be a cloning or expression library. The kit or library may comprise a plurality of vials, wells, plates or tubes providing the plurality of spatially separated agents. For example, a kit may be provided with individual vials containing expression plasmids that encode individual polypeptides in frame with a common cloning site. The contents of each vial may be mixed with exonucleases known in the art to cleave at desired nucleic acid bonds to produce a linearised vector that can be annealed with a nucleic acid fragment encoding e.g., a nanobody or antibody fragment sequence which targets a protein of interest. The kit may thus comprise spatially separated expression plasmids for a plurality of fusion polypeptides, e.g. fusion polypeptides comprising an affinity ligand fused to a peptide mass tag. Introduction of each expression plasmid into an expression host thus allows for a plurality of fusion polypeptides to be produced.

### Method of Analysing a Target Protein

The invention also relates to a method of analysing a target protein using a peptide mass tag as described herein. The method involves using a polypeptide (comprising an affinity ligand and a peptide mass tag, said peptide mass tag comprising a dipeptide with fragmentation propensity) as a “hook” to bind to a target protein within a sample. The affinity ligand binds to the target protein, producing a complex with the target protein. This “polypeptide complex”, comprising the polypeptide and the target protein, can then be “fished out” of sample using a capture agent. The polypeptide complex thus obtained can be recovered, and analysed by a mass spectrometric method. The method involves steps (i) to (vi), which are discussed in more detail hereafter.

Two or more of the steps may be performed simultaneously. For instance, steps (i) and (ii) may be performed simultaneously where the sample is provided and simultaneously exposed to the polypeptide. The mixture may further simultaneously be exposed to a capture agent, as in step (iii). More typically, the above steps may be performed sequentially, with (i) preceding (ii), (ii) preceding (iii), (iii) preceding (iv), (iv) preceding (v), and (v) preceding (vi).

One or more of the steps may be repeated. By way of example, step (iii) may be repeated by exposing the first composition to a capture agent more than once. Similarly, step (iv) may be repeated by repeating the exposure of the capture agent to a means for recovering the polypeptide complex.

It is envisaged that one or more of the steps may be repeated using differing reagents. By way of example, step (iii) may be performed with a first capture agent and may be repeated with a second, differing, capture agent. In another example, steps (i) and (ii) may be repeated by providing a plurality of different samples each comprising a target protein, and exposing each sample to a polypeptide comprising an affinity ligand for the target protein fused to a peptide mass tag, said peptide mass tag comprising a dipeptide with fragmentation propensity. These repeated steps may be performed simultaneously or sequentially.

### Step (i)

Step (i) involves providing:
a sample comprising the target protein, and
a polypeptide comprising an affinity ligand for the target protein fused to a peptide mass tag, said peptide mass tag comprising a dipeptide with fragmentation propensity.

The affinity ligand is as described herein.

As regards the peptide mass tag, that tag may or may not comprise a binding moiety as described herein. Generally, the peptide mass tag does comprise a binding moiety (as described herein). In such cases, the binding moiety is generally a moiety that can bind to the capture agent. Thus, the peptide mass tag provides both the “hook” (that is, the affinity ligand) to attach to the target protein, and the “rod” (that is, the binding moiety) by which the polypeptide complex can be pulled out of the sample. In such cases, the peptide mass tag is a peptide mass tag as described in detail above. Accordingly, in such cases, step (iii) comprises exposing the first composition to the capture agent to reversibly bind the binding moiety to the capture agent.

In other cases, it may not be necessary for the peptide mass tag to include a binding moiety. This is possible where the target protein itself may bind to the capture agent, meaning that the target protein may act as the “rod” by which the polypeptide complex can be retrieved from the sample. In such cases, the peptide mass tag is as described above, except that it does not require a binding moiety. Thus, the peptide mass tag utilised in the method can exclude the binding moiety described herein. Typically, though, the peptide mass tag includes a binding moiety and is as described in detail above.

Regardless of whether the peptide mass tag includes or excludes a binding moiety, the peptide mass tag does include a dipeptide with fragmentation propensity as described herein.

In the polypeptide of step (i), the affinity ligand is fused to the peptide mass tag. By “fused” is meant that the two are bound together. Generally, the affinity ligand is covalently bound to the peptide mass tag.

### Step (ii)

Step (ii) comprises exposing the sample to the polypeptide. This step may simply comprise adding the polypeptide to the sample. For instance, step (ii) may comprise mixing the polypeptide with the sample. Generally, the sample is a liquid sample and the polypeptide is provided in a liquid composition, and so step (ii) may comprise mixing these liquids together.

When the polypeptide and the sample are mixed, the affinity ligand of the polypeptide binds to the target protein (assuming that the target protein is present). This produces a “polypeptide complex”, the polypeptide complex comprising the polypeptide in complex with the target protein. The resulting composition, produced by exposing the sample to the polypeptide, is referred to as a “first composition”. The first composition is typically a liquid composition. Typically, the first composition is aqueous.

If step (ii) is repeated (for instance with a plurality of samples), more than one “first composition” may be produced. These may conveniently be numerically labelled. For instance, the method may involve, in step (i), providing a plurality of samples each comprising the target protein, said samples being referred to as “sample 1”, “sample 2”, and so on.

In such cases, step (ii) may comprise exposing each sample to the polypeptide. Consequently, step (ii) may produce a “first composition 1” obtained from sample 1; a “first composition 2” obtained from sample 2; and so on. More commonly, though, where it is intended to utilise multiple samples, those samples are combined in or prior to step (i).

If a plurality of first compositions are produced, they may be combined prior to step (iii).

Step (ii) may involve one or more processes to increase the proportion of the target protein which is bound to the polypeptide. For instance, step (ii) may comprise incubating the polypeptide together with the sample.

### Step (iii)

Step (iii) involves exposing the first composition to a capture agent. The capture agent is a species which can capture the polypeptide complex from the first composition, and can release the polypeptide complex to form the second composition. Thus, the capture agent is a species which can reversibly bind to the polypeptide complex.

The capture agent is capable of binding to the binding moiety of the peptide mass tag or, less preferably, to the target protein. The nature of the capture agent will depend on the nature of the binding moiety or, if appropriate, the target protein.

The capture agent is typically capable of forming covalent and/or non-covalent bonds to the said binding moiety or target protein. Preferably, the capture agent binds to the polypeptide moiety with high affinity. It is therefore preferred to use a capture agent which specifically binds the polypeptide, and does so with a high binding efficiency. Ideally, the capture agent binds at least 50% of the polypeptide from the first composition; preferably at least 90%; most preferably at least 95%.

Thus, the capture agent generally comprises a moiety which specifically binds to the polypeptide complex with high affinity. Such a moiety may be an antibody; an antibody derivative; or an antigen-binding fragment thereof, as described above in connection with affinity ligands. The moiety of the capture agent may be any other high affinity binding agent described above for use as an affinity ligand. Suitable moieties can be readily selected by the skilled person.

Additionally, the capture agent must be able to release the polypeptide complex (as in step (iv). This can conveniently be accomplished where the capture agent comprises a solid; in that case, the capture agent bound to the polypeptide complex can be moved from one environment to another, to facilitate the removal of the polypeptide complex. Preferably, therefore, the capture agent comprises a solid substrate. Suitable examples of solid substrates include particles, granules, solid surfaces, and so on.

Suitably, therefore, the capture agent may comprise any of: an antibody; an antibody derivative; or an antigen-binding fragment thereof, bound to a substrate. Preferably the substrate is a solid substrate.

A particular example of the capture agent is an affinity resin. An affinity resin may be any resin used in affinity chromatography, including both selective and non-selective resins, including covalently-coupled resins. Most preferably the capture agent is a selective affinity resin compatible with competitive elution (e.g. antibody resins [such as 1D4, anti-FLAG, STREPII,]), and/or with recovery by protease cleavage. A non-selective resin may be a protein A, G, or L, lectin, or heparin binding resin. Such resins target classes of proteins with similar bonding capabilities.

When the first composition is exposed to the capture agent, the polypeptide complex reversibly binds to the capture agent. Generally, the capture agent comprises a solid, and so binding of the polypeptide complex to the capture agent involves immobilising the polypeptide complex on a solid capture agent.

As the first composition is generally a liquid, step (iii) typically involves passing a liquid first composition over a solid capture agent. For instance, step (iii) may involve passing the first composition through a column, wherein the column contains a solid capture reagent. However, many variations of the capture process are possible; for instance, step (iii) may involve passing the first composition over a bed comprising the capture agent or through a chip or channel comprising the capture agent. In a particular aspect, the capture agent may be an affinity resin and step (iii) involves passing the liquid first composition through a chromatographic column including the affinity resin.

Step (iii) may be repeated. For instance, the first composition may be passed over more than one capture agent, or may be passed over the capture agent more than once.

Following step (iii), the target protein (in the form of a complex with the polypeptide) is bound to the capture agent. The binding typically occurs between a binding moiety of the peptide mass tag and the capture agent, but may alternatively involve the target protein itself. The binding may further alternatively be between the affinity ligand and a capture agent specific for the affinity ligand.

After step (iii), the capture agent (to which the polypeptide complex is reversibly bound) may be washed. This can remove any contaminants from the first composition, other than the desired polypeptide complex, which remain associated with the capture agent. The optional washing step is typically performed at the end of step (iii) and before step (iv). The washing may be performed with a suitable liquid, such as water or a buffer solution. Typically, the washing does not detach the polypeptide complex from the capture agent.

### Step (iv)

Step (iv) involves recovering the polypeptide complex from the capture agent. By “recovering” is meant that the polypeptide complex is detached from the capture agent, under non-denaturing conditions. Thus, the polypeptide complex is recovered from the capture agent intact. Preferably, in the recovered polypeptide complex, the target protein remains in its native state. The composition comprising the polypeptide complex which has been detached from the capture agent is referred to as the “second composition”. The second composition differs from the first composition in that, although it contains the polypeptide complex, it contains few or no other elements from the sample. Preferably, the second composition is free of protein components from the sample other than the target protein (typically in the form of the polypeptide complex).

As the capture agent typically comprises as solid, step (iv) generally involves flowing a liquid over the capture agent to recover the polypeptide complex. Step (iv) generally comprises displacing the polypeptide complex from the capture agent by exposing the capture agent to a solution, the solution comprising a competitive agent which competes with the polypeptide complex to bind with the capture agent. This process can be referred to as a competitive elution. Accordingly, step (iv) may involve recovering the polypeptide composition comprising the polypeptide complex from the capture agent by competitive elution, using a solution comprising a competitive agent. Alternatively, step (iv) may involve recovering the polypeptide composition comprising the polypeptide complex from the capture agent by protease cleavage (typically where the peptide mass tag includes a protease cleavage site, as discussed above), using a solution comprising a protease.

The competitive agent may include the binding moiety from the peptide mass tag. The competitive agent may be the polypeptide itself. However, the competitive agent may have a different binding motif.

In addition to a competitive agent, the solution used in competitive elution may comprise a mass-spectrometry compatible buffer.

Step (iv) may involve flowing said solution over the capture agent. Alternatively or additionally, step (iv) may involve incubating said solution with the capture agent.

Step (iv) produces a composition comprising the polypeptide complex, recovered from the capture agent. This composition is referred to as the “second composition”. The second composition is typically a liquid composition. The second composition may also comprise a competitive agent, and/or a mass spectrometry compatible buffer. Preferably, the second composition is a liquid composition comprising the polypeptide complex, a competitive agent or protease, and a mass spectrometry compatible buffer.

Step (iv) may be repeated one or more times. For instance, the competitive elution may be repeated and the resulting solutions may be combined to produce the second composition. For instance, the capture agent may be exposed to a plurality of solutions, each comprising a competitive agent, to produce a plurality of solutions each comprising the polypeptide complex (and, generally, a competitive agent). These solutions may be combined to produce the second composition.

Generally the second composition is aqueous.

The second composition may comprise the polypeptide complex at a low concentration, especially where the second composition is produced by combining several solutions following competitive elution. Accordingly, at the end of step (iv), the second composition may be enriched. By “enriched” is meant that the concentration of the polypeptide complex is increased. The resulting composition may be referred to as an “enriched second composition”. Thus, reference to a “second composition” herein includes an “enriched second composition”. The enrichment step may be performed at the end of step (iv), before step (v).

The second composition may be enriched by, for instance, evaporating water from the second composition or by concentration via molecular weight cutoff filtration devices.

The optionally enriched second composition comprises the polypeptide complex, largely isolated from the other components of the original sample. Consequently, the second composition is suitable for analysis by mass spectrometry. Accordingly, in further steps (v) and (vi), the second composition.

### Step (v)

Step (v) involves ionising the second composition. The ionisation produces gas-phase ions, and in particular a “tagged gas-phase ion”. As all ions produced in step (v) are necessarily gas-phase ions, and so the term “gas-phase” can be omitted for convenience. Thus, the “tagged gas-phase ion” may be referred to herein as a “tagged ion”.

A tagged gas-phase ion comprises the polypeptide complex (as defined above). Any ionization method that has the capacity to produce tagged gas-phase ions would be suitable to perform the invention (that is, any ionisation process which does not itself fragment the complex). The ionisation process is generally performed by some form of electrospray ionisation, preferably by nanoelectrospray ionisation or by static electrospray ionisation. Such ionisation methods include native electrospray ionisation.

In a nanoelectrospray ionisation process, nano-sized droplets are produced. This can be achieved by routine adjustment of the electrospray conditions, for example, using electrospray capillary emitters (particularly those that have been drawn to a tip with a diameter of less than 10 μm, preferably <5 μm, most preferably between 0.5-2 μm).

In a preferred embodiment, the ionisation process of step (v) is a native electrospray ionisation process. In a native electrospray ionisation process, the conditions during the electrospray procedure are kept close to physiological conditions. In particular, the pH of the sample during the electrospray process is maintained at a physiological pH. The pH is typically from 5 to 8. In such conditions, the native conformation of the intact target protein may be optimally preserved.

The tagged gas-phase ion may be suitable for analysis by mass spectrometric methods. In practice, a number of further steps are often performed in order to confine and isolate the tagged gas-phase ion before step (vi) is performed. These steps are described below.

In order to perform any process on the tagged ion, it is generally necessary to confine it. By “confined” it is meant that the tagged ion is localised within an electromagnetic field. This localisation may be around a point, or along an axis or some curvilinear path. Confinement indicates bounding of the ion motion and location in either two or three dimensions. A tagged gas-phase ion which has been confined may be referred to as a “confined tagged gas phase ion”, the tagged gas phase ion itself being defined as above.

Any suitable apparatus may be used to confine the tagged ion (and/or any ion derived therefrom). Suitable examples of apparatus which may be used to confine these species include a mass filter (such as a quadrupole mass filter); a radio frequency ion guide (such as a radio-frequency two dimensional multipole ion guide, a radio-frequency stacked ring ion guide, or a radio frequency stacked ring ion funnel), or an ion trap (a typical example being a dual-pressure radio frequency quadrupole linear ion trap). One example is a radio frequency multipole ion guide (ion routing multipole) which can be operated as an ion guide or an ion trap by adjusting the magnitudes of the radio frequency (RF) and direct current (DC) potentials applied to its electrodes.

The tagged ion may be confined in a mass spectrometer. Thus, one step performed after (v) may be the transfer of the tagged ion to a mass spectrometer. In such a process, subsequent to ionisation the tagged gas-phase ion is transported into the vacuum of the mass spectrometer by passing the tagged gas-phase ion through a heated capillary tube or a suitably small orifice into a region at a pressure much below atmospheric pressure. For static electrospray ionisation and nanoelectrospray ionization the emitter tip is located within a few millimetres of the capillary entrance or orifice. For instance, the process may involve passing the tagged gas-phase ion through a capillary tube heated to a temperature of greater than 250° C., for instance from 250 to 450° C., e.g. from 300 to 400° C. These high temperatures assist with the removal of residual solvent molecules without denaturing the polypeptide complex.

A mass spectrometer with an appropriately configured nano electrospray ion source may be used to perform the ionisation step. A suitable example is a Thermo Scientific™ Orbitrap Eclipse™ Tribrid™ mass spectrometer with Thermo Scientific™ NanoSpray Flex NG™ ion source configured for static native electrospray ionisation. This instrument utilizes a heated metal capillary atmosphere to vacuum interface.

In addition to the polypeptide complex, the tagged ion may comprise non-specifically bound molecules such as residual solvent molecules and/or lipid molecules. The presence of these species can generate unwanted complexity in any eventual mass spectrum and can dilute the intensity of the mass peak(s) associated with the tagged ion. It is therefore desirable to remove them. Preferably, prior to step (vi), the tagged ion is largely or entirely free of non-specifically bound molecules.

As noted in the preceding paragraph, these non-specifically bound molecules can be lost during the confinement of the tagged ion. However, a further step may be desired in order to produce a tagged ion which is largely or entirely free of non-specifically bound molecules. Accordingly, one step which may be performed after ionisation in step (v) is the removal of non-specifically bound molecules.

Removal of non-specifically bound molecules may be achieved using techniques disclosed in the art. For instance, the confined tagged ion may be exposed to a collision gas. Collisions of the confined tagged ion with the collision gas can promote the loss of non-specifically bound molecules. A collision gas is generally a noble gas such as Neon, Argon, or Xenon or a relatively non-reactive molecular gas such as nitrogen (N₂) or sulfur hexafluoride (SF₆) with generally Nitrogen or Argon being preferred. The pressure of collision gas surrounding the confined ion in such cases may be, for instance, 1-10 mTorr.

Recently, it has also been found that exposure of confined ions to infra-red irradiation may also be used to remove non-specifically bound molecules.

Another step which it may be useful to perform is the mass isolation of the tagged ion.

In practice, the first composition generally contains a large number of proteins, some or all of which may be the target protein. Moreover, although the affinity of the polypeptide for the target protein is large, the first composition may comprise some target protein which is not bound to the polypeptide. Accordingly, ionising the sample as in step (v) typically produces a large cloud of gas phase ions. This may include ions comprising proteins other than the target protein.

In particular, the ionisation step produces a plurality of tagged ions, each comprising a polypeptide complex as described herein. However, each tagged ion within that plurality of tagged ions may initially differ in mass, due to the presence of differing numbers of non-specifically bound molecules (such as solvent molecules).

Further, upon ionisation of the sample, the produced plurality of tagged ions will have a distribution of charge states. Each tagged ion within the said plurality may be ionized to any one of a range of charge states (even where each tagged ion is comprised of an target protein that has the same amino acid sequence, or is chemically identical). For instance, the plurality of tagged ions may comprise two or more tagged ions, wherein each tagged ion has differing charge state.

Thus, in practice, the population of tagged ions typically produced by ionisation in step (v) can often have a broad m/z distribution.

Fortunately, the confinement of ions often allows specific ion m/z ranges to be selected such that ions having m/z outside of the selected ranges are not confined and hence are eliminated. All RF ion confinement devices have m/z dependence in the confinement of ions. RF quadrupole field devices such as RF quadrupole m/z filters and RF quadrupole linear ion traps can be operated to have very sharp transitions between ranges of m/z confinement and m/z ejection or non-confinement. The numerous techniques for effecting selection of specific m/z ranges with RF quadrupole field devices are well known in the art.

Depending upon the specific device, the range for m/z selection may be narrower than 0.1% of the m/z at the midpoint of the selected m/z range. Selection of a range of mass-to-charge ratios, if performed on an ion beam is often referred to in the art as “mass selection” and, with somewhat more rigor, as “m/z selection”. Selection of a specific mass-to-charge ratio, or range of mass-to-charge ratios and elimination of ions having m/z values outside of that selected range when performed on a trapped population of ions is often referred to in the art as “mass isolation” and, with somewhat more rigor, as “m/z isolation”. For the purposes of this specification m/z isolation and m/z selection can be considered equivalent terms.

Performing an m/z isolation step allows the isolation and hence analysis of a tagged ion having a specific m/z ratio, or falling within a specific m/z range. The ability to ability to m/z select an ion or group of ions to be subjected to further transformational processes allows more detailed analysis of the structure and composition of the polypeptide complex therein. After the production of any ion, the process may comprise a step of performing m/z isolation in order to isolate the ion or ions having a particular m/z or range of m/z values.

m/z selection of an ion can be performed, for instance, in an RF ion guide and with much more precision in an RF quadrupole linear ion trap analyser or a quadrupole m/z filter. The ions having unwanted m/z ratios may be ejected from the RF ion guide, RF quadrupole linear ion trap or RF quadrupole m/z filter. This may comprise actively adjusting the magnitudes and frequency composition, or, in some instances, applying additional the types of voltages to the RF ion guide, RF quadrupole linear ion trap or RF quadrupole m/z filter electrodes in order to expel particular m/z ranges of ions, or may simply comprise allowing the ions having unwanted m/z ratios to escape.

In practice, the process involves pre-determining the desired range of m/z ratios to be retained, and then ejecting the ion(s) having an m/z ratio outside that range from an RF ion guide, RF quadrupole linear ion trap, RF quadrupole m/z filter or other device in which the ion is confined.

For instance, in some embodiments after step (v) the method described herein may involve confining the tagged ion in an ion trap (the term “ion trap” being used to refer to any suitable confinement device) and isolating the tagged ion by ejecting ions outside one or more pre-determined m/z windows from the ion trap.

This m/z selection step can of course be repeated prior to and/or after the ion transformative process(es) performed on the tagged ion and the ion(s) derived therefrom, described hereafter.

In summary, step (v) produces a tagged gas-phase ion which is preferably largely or entirely free of non-specifically bound molecules. Particularly preferably, the tagged gas-phase ion consists only of the complex comprising the polypeptide and the target protein. The tagged gas-phase ion may also preferably be isolated. To achieve this, at the end of step (v) and before step (vi), a number of optional additional steps may be performed. These steps include one or more selected from the following, in any order (although step (a) typically occurs before either of (c) or (d)):
(a) confinement of the tagged ion;
(b) transfer of the tagged ion to a mass spectrometer;
(c) removal of non-specifically bound molecules, such as residual solvent or lipid molecules; and
(d) m/z isolation of the tagged ion.

Subsequently, in step (vi), the tagged gas-phase ion may itself be detected or alternatively may be subjected to an ion transformative process, allowing a product ion obtained from the tagged gas-phase ion to be detected.

### Step (vi)

Step (vi) involves m/z analysing and detecting the tagged gas-phase ion, and/or an ion derived therefrom.

By “m/z analysing” is meant that the ion is subjected to an m/z-dependent dispersion process. The process allows the mass-to-charge ratio of the detected ion to be determined. The process generally disperses the ion in space, or in time, or in frequency of motion, dependent on the ion's m/z. This means that when the ion is detected, its m/z may be determined with reference to its location of detection, its time of arrival at the detector, path stability as determined by the applied voltages to the m/z analyser electrodes or the frequency of its motion indicated by the frequency of the a detected signal it induces. The m/z dependent dispersion process typically involves an interaction with an electromagnetic field. One example involves the interaction of an ion with an electric field within a time-of-flight mass spectrometer. In that case, the ion is given kinetic energy by an electric field, and is thus accelerated to a velocity dependent on its m/z. Consequently, the time of flight required to arrive at the detector after interaction with the electric field depends on the ion's m/z and allows its m/z to be determined.

Thus, step (vi) comprises determining the m/z of the tagged ion and/or an ion derived therefrom.

In practice, the method typically comprises measuring signal intensity as a function of mass-to-charge (m/z) ratio, by any suitable measurement means. Any suitable combination of m/z analyser and detector may be used. By way of illustration, any of the following could be used:
a high field orbitrap analyser;
an RF quadrupole linear ion trap analyser and its associated conversion dynode-electron multiplier based ion detector in the same instrument;
a Time-of-flight analyser and associated ion detector; or
a Fourier Transform Ion Cyclotron Resonance (FT-ICR) analyser.

Performing step (vi) will typically produce an m/z spectrum, or m/z spectral data. The m/z spectrum may be analysed to determine the m/z of any ion produced by the process described herein.

In its simplest aspect, therefore, step (vi) may comprise simply m/z analysing and detecting the tagged gas-phase ion. Detecting the presence of the tagged ion can provide useful information, in that it serves to confirm that the target protein was present in the sample.

However, often it is necessary to perform processing in order to extract useful information from the process. The peptide mass tag is invaluable for this process. In particular, step (vi) may comprise utilising the peptide mass tag to identify one or more characteristics of the target protein, such as its mass. This is particularly useful in cases where the precise identity of the target protein is unknown. In such cases, the process is as follows.

The sample (and hence the first composition) typically contain a large number of differing components. Consequently, the ionisation performed in step (v) generally produces an ion cloud containing ions having a broad range of m/z ratios. It may not be known which m/z ratio within this broad range corresponds to the tagged ion. However, the peptide mass tag allows straightforward identification of the relevant peak. The peptide mass tag comprises a dipeptide with fragmentation propensity. The peptide mass tag may be divided conceptually into two parts, across that dipeptide: the “target-binding portion”, comprising the target protein, the affinity ligand, the amino acid of the dipeptide with fragmentation propensity proximal to the affinity ligand (that is, bound directly or indirectly to the affinity ligand), and any intervening amino acid(s); and the “reporter portion” comprising the other amino acid of the dipeptide with fragmentation propensity and the remainder of the peptide mass tag. Consequently, fragmentation at the said dipeptide generates at least two fragments. Typically, one fragment comprises or consists of the target protein and the target-binding portion of the peptide mass tag (unless this has fragmented further under the relevant conditions). The mass of this portion is largely dependent on the mass of the target protein and is typically much larger than 10 kDa. Often, the mass of this portion will be in the range of 50-1000 kDa.

Another fragment comprises or consists of the reporter portion. This portion typically has a lower mass. The mass of the reporter portion is typically in the range of about 2 kDa to 8 kDa, preferably about 3 kDa to about 5 kDa. This mass range is well separated from the masses of other species typically found in the mass spectrum. The reporter portion is therefore easily detectable.

Accordingly, the detection of the reporter portion clearly signals the presence of the target protein. Even if the target protein fragments or cannot easily be distinguished, the presence of the reporter portion can confirm that the target protein is present and has bound to the polypeptide.

Typically, when the reporter portion is generated from the tagged ion, the reporter portion is charged. Generally, therefore the reporter portion is ionised and may equally be referred to as a reporter ion. That is, the reporter ion comprises the remainder of the peptide mass tag after loss of the affinity ligand (and the target protein bound thereto), the amino acid of the dipeptide with fragmentation propensity proximal to the affinity ligand, and any intervening amino acid(s).

Similarly, when the target-binding portion is generated from the tagged ion, the target-binding portion is typically charged and may be referred to as a target-binding ion.

Thus, in a useful embodiment step (vi) comprises analysing and detecting the reporter portion of the polypeptide.

For example, steps (v) and (vi) may involve:
(v)(a) ionising the second composition to produce a tagged gas-phase ion, the tagged gas-phase ion comprising the polypeptide complex;
(v)(b) mass isolating the tagged gas-phase ion;
(vi)(a) fragmenting the tagged gas-phase ion to produce a reporter ion; and
(vi)(b) m/z analysing and detecting the reporter ion.

Of course, it may not be known which m/z ratio in the distribution produced by ionisation in step (v)(a) relates to the polypeptide complex. Consequently, steps (v)(b) to (vi)(b) may be repeated in turn, isolating different m/z ratios in turn, until the m/z ratio which yields the reporter ion is identified.

It should be appreciated that this detection of the reporter ion fragment can provide more information than simply identifying which m/z species produced by step (v) comprises the target protein. In an aspect, the method relates to a particularly useful “multiplexing” process, described in more detail below. Briefly, in such cases, a number of different samples each believed to comprise the target protein are each exposed to differing polypeptides. The polypeptides are isobaric but differ in the arrangement of amino acids across the dipeptide with fragmentation propensity, such that the chemical identity of the reporter ion in each case differs. Preferably, the mass of each reporter ion differs. Thus, each sample is associated with a differing reporter ion. The samples may be combined to produce a single first composition, in order to maximise the quantity of polypeptide complex in the first composition. When step (v) is performed as above, the m/z species isolated will comprise the tagged gas-phase ion obtained from each different sample, as each will have the same mass. However, when step (vi) is performed as above, the polypeptide complex originating from each different sample will yield a different reporter ion. Thus, m/z analysing and detecting the reporter ions reveals which sample(s) contained the target protein, and potentially also indicates the relative abundance of the target protein in each sample.

The above-described aspect illustrates an important feature of step (vi), which is that the m/z analysis procedure may be performed on ions produced directly by step (v), or on ions derived therefrom. Thus, step (vi) may involve performing an “ion transformative process” on an ion (such as the tagged ion) in order to generate a product ion, and then subsequently m/z analysing that product ion. This may be repeated a number of times.

The manipulation of ions produced by step (v) is particularly useful where some structural feature (or even the identity) of the target protein is unknown. In that case, it is advantageous to subject the tagged ion and/or one or more product ion(s) derived therefrom to one or more ion transformative processes (such as fragmentation or charge reduction). It is then possible to m/z analyse and detect the various product ions thus derived from the tagged ion. Such processes, when performed on a population of chemically identical tagged ions with identical structure and molecular composition, can yield information about the structure and chemical identity tagged ion.

Thus, in a preferred embodiment of step (vi), the tagged ion is subjected to one or more ion transformative processes to produce at least one product ion, and the product ion is m/z analysed and detected. A “product ion” refers to an ion produced from another ion. A product ion is produced by an ion transformative process, performed on another ion. In this context a first-generation product ion is an ion which is produced directly from a tagged ion. The first-generation product ion may be the reporter ion. Other possible first-generation product ions include the target-binding portion, or a fragment thereof.

In other preferred embodiments of step (vi) the product ion may be subjected to one or more ion transformative processes to produce one or more next-generation product ion(s). Any product ion (that is, any product ion derived from a previous generation product ion), may be m/z analysed and detected. For instance, tagged ion may be fragmented to produce a product ion which is the target-binding portion, and this first-generation product ion may be subject to further fragmentation though one or more ion transformative processes. Any next-generation product ion formed through this succession of transformative processes may be m/z analysed and detected.

Thus, step (vi) may comprise m/z analysing and detecting the tagged ion, and/or an ion derived therefrom, in a tandem mass spectrometry method.

In reality, the process is typically performed on a plurality of tagged ions, and a plurality of first-generation product ions is typically produced. Similarly, subsequent ion transformation processes typically produce a plurality of next-generation product ions. Each product ion in the plurality of next-generation product ions may be the same or different.

Accordingly, by way of illustration, step (vi) may comprise fragmenting the tagged ion to produce a product ion which is a fragment ion (typically the reporter ion or the target-binding ion). This fragment ion may then be directly m/z analysed and detected. Alternatively, this fragment ion may be charge-modified prior to m/z analysis and detection. For instance, the charge of a fragment ion may be reduced in order to increase its m/z ratio, as this can increase the separation in m/z between product ions that may be generated when a population of chemically identical tagged ions are simultaneously transformed according to step (vi) of the method. Where the fragment ion is the reporter ion, this is often unnecessary as the mass of the reporter ion is typically low and hence its charge is generally also low. However, where the fragment ion is the target-binding portion (or a part thereof), charge-modification may well be of interest.

Thus, step (vi) may comprise subjecting the tagged ion to one or more ion transformative processes to produce a product ion (specifically, a first-generation product ion). This first-generation product ion may then be directly m/z analysed and detected. In another alternative, a fragment ion may itself be fragmented, to produce a further fragment ion, of the next generation. For instance, the first-generation product ion may be subjected to one or more further ion transformative processes, to produce a second generation product ion. The second-generation product ion can be detected or be subjected to further ion transformative processes. That is, more than two ion transformative processes may be performed during step (vi). For example, 3, 4, 5, 6, 7, 8, 9 or 10 ion transformative processes may be performed during step (vi).

Multiple fragmentation processes will lead to large numbers of fragment ions. Accordingly, where fragmentation processes are performed, m/z isolation of a desired ion or several ions is generally performed between fragmentation processes and also optionally prior to m/z analysis and detection. The m/z isolation process is discussed above.

However, the ion transformative processes need all not necessarily be fragmentation processes. A wide variety of ion transformative processes (particularly fragmentation processes) are envisaged for any fragmentation process or processes performed during step (vi). For instance, the dissociative ion transformation may be achieved by one or more of the following methods: infrared multiphoton dissociation (IRMPD), electron transfer dissociation, activated ion electron transfer dissociation, electron capture dissociation, UV photodissociation and collision-induced dissociation.

Collision-induced dissociation is typically effected using a noble gas such as Helium, Neon, Argon, or Xenon or a relatively non-reactive molecular gas such as nitrogen (N₂) or sulfur hexafluoride (SF₆). The pressure of collision gas used may be, for instance, 1-20 mTorr or greater.

The selection of the most appropriate ion transformative process during step (vi) may depend on the type of product (e.g. a fragmentation product) desired. For instance, some fragmentation techniques are better-suited to breaking non-covalent bonds. Such techniques include, IRMPD, collision-induced dissociation (including RF Ion Trap type resonant collision induced dissociation, and beam type/collision cell type low energy collision induced dissociation). Surface induced dissociation is also an alternative, although less preferred.

By contrast, where it is intended to dissociate covalent bonds, suitable techniques include IRMPD, collision-induced dissociation (including trap-type CID, and low-energy beam-type CID), ultra-violet photodissociation (UVPD) and electron transfer dissociation.

It may be desirable to utilize some form of fragment ion protection (e.g. product ion parking, see US 2010/0084548 and Ugrin et al., Journal of the American Society of Mass Spectrometry, 2019, vol. 30 U.S. pp2163-2173, which are incorporated herein by reference) to limit the extent of generation of internal (non-C- and N-terminal including products) where IRMPD, UVPD or ETD are employed.

Ion transformative processes include charge modification processes. Charge reduction is generally effected by proton transfer ion-ion reactions, but it is possible (although less preferred) to achieve this by other means such as (but not limited to) electron transfer ion-ion reactions or electron capture (ion-electron) reactions.

Thus, to summarise the above in general terms, step (vi) may comprise:
(vi)(a) subjecting the tagged gas-phase ion to an ion transformative process to produce a first-generation product ion; and
(vi)(b) m/z isolating and optionally m/z analysing and detecting the first-generation product ion.

The term “m/z selecting” may be used instead of “m/z isolating”. If the first-generation product ion is to be further fragmented, it will generally not be detected. If the first-generation product ion is to be detected, then step (vi) may comprise:
(vi)(a) subjecting the tagged gas-phase ion to an ion transformative process to produce a first-generation product ion; and
(vi)(b) m/z analysing and detecting the first-generation product ion.

Of course, in such cases the first-generation product ion may also be mass isolated prior to m/z analysis and detection.

The first-generation product ion may often be the reporter ion. Thus, in a preferred embodiment, the polypeptide complex comprises:
a target-binding portion comprising the target protein, the affinity ligand, the amino acid of the dipeptide with fragmentation propensity bound to the affinity ligand, and any intervening amino acid(s); and
a reporter portion comprising the other amino acid of the dipeptide with fragmentation propensity and the remainder of the peptide mass tag;
   and step (vi) comprises:
(vi)(a) subjecting the tagged gas-phase ion to an ion transformative process to produce a reporter ion which comprises or preferably consists of the reporter portion; and
(vi)(b) m/z isolating and optionally m/z analysing and detecting the reporter ion.

Alternatively, step (vi) may comprise:
(vi)(a) subjecting the tagged gas-phase ion to an ion transformative process to produce a target-binding ion which comprises or preferably consists of the target-binding portion; and
(vi)(b) m/z isolating and optionally m/z analysing and detecting the target-binding ion.

Instead of being detected, the first-generation product ion may be further manipulated prior to detection. For instance, after steps (vi)(a) and (vi)(b), step (vi) may additionally comprise:
(vi)(c) subjecting the first-generation product ion to a further ion transformative process to produce a second generation product ion; and
(vi)(d) m/z isolating, and/or m/z analysing and detecting, the second generation product ion.

Although the reporter ion is generally of low mass, it may be advantageous to perform at least steps (vi)(c) and (vi)(d) to fragment the reporter ion in order to determine its structure (for instance, its amino acid sequence). This is particularly useful in the multiplexing aspect of the invention described herein, wherein the process is performed using a number of different samples each tagged with a polypeptide comprising a differing reporter ion. Generally, the reporter ions associated with each sample may have a different mass. However, the reporter ions may alternatively or additionally differ from one another by the order of the amino acids therein, or by the location of a post-translational modification. In such cases, it may be necessary to fragment the reporter ion(s) detected in order to deduce all or part of their chemical structure (such as the order or amino acids therein, or the location of post-translational modifications) from the fragments thus obtained. Such sequence information can be obtained using commercial software.

Thus, in some cases, step (vi) may comprise:
(vi)(a) subjecting the tagged gas-phase ion to an ion transformative process to produce one or more reporter ion(s);
(vi)(b) m/z isolating reporter ion(s);
(vi)(c) subjecting the reporter ion(s) to a further ion transformative process to produce one or more second generation product ion(s); and
(vi)(d) m/z isolating, and/or m/z analysing and detecting, the second generation product ion(s).

Of course, further isolation and fragmentation processes may be performed, and labelled (vi)(e), (vi)(f), and so on. By way of example, step (vi) may comprise:
(vi)(a) subjecting the tagged gas-phase ion to an ion transformative process to produce one or more reporter ion(s);
(vi)(b) m/z isolating reporter ion(s);
(vi)(c) subjecting the reporter ion(s) to a further ion transformative process to produce one or more second generation product ion(s);
(vi)(d) m/z isolating one or more of the second generation product ion(s);
vi)(e) subjecting the isolated second generation product ion(s) to a further ion transformative process to produce one or more third generation product ion(s); and
(vi)(d) m/z isolating, and/or m/z analysing and detecting, the third generation product ion(s).

The process may further comprise deducing the identity of the reporter ion(s).

Similarly, the series of fragmentation steps described above with respect to the reporter ion(s) may be performed on the target binding ion (or fragment thereof) produced by step (vi)(a). Instead of yielding information on the structure of the reporter ion(s), such processes may yield information on the structure of the target binding ion(s). In particular, such processes may yield information on the structure of the target protein.

It is clear, therefore, that the method is applicable to a wide variety of situations and so the information obtained by the analysis in step (vi) is similarly variable.

### Multiplexing Method

In a particularly useful aspect, the method described herein can be performed upon a number of differing target proteins or samples combined together. This aspect may be referred to as the “multiplexing” process.

In such cases, the first composition comprises a plurality of target proteins. The target proteins may differ, chemically. This embodiment may have the advantage of ease and convenience, allowing differing proteins to be analysed together. However, the differing proteins will have different masses and so will typically appear at different regions of the m/z spectrum, meaning that the advantages of this embodiment are somewhat limited.

More usually, in the multiplexing embodiment, the first composition comprises a plurality of target proteins wherein each target protein is the same protein. The “same protein” may encompass differing proteoforms and differing degrees of post translational modifications. More preferably, though, each protein has the same amino acid sequence and identical post translational modifications (that is, each protein is the same proteoform), as these will have the same mass.

Typically, the plurality of target proteins is obtained by combining different source samples, each believed to contain the target protein (and preferably each containing the target protein). Each “source sample” is a sample as described herein. The difference is simply that these “source samples” are combined before being utilised in the process of the invention. Accordingly, for instance, each source sample may be obtained or obtainable from a tissue, biofluid or cell.

In such cases, step (i) involves providing a number of different source samples. Generally, each source sample is believed to comprise the target protein. Thus, each source sample is typically the same type of sample (for instance, each source sample generally comprises the same type of biological material). However, the source samples may differ in that they may each be taken from different patients; or they may be taken from the same patient at different times, or they may be taken from different tissues of the same patient, or they may be the same biological material each exposed to different conditions (such as different pharmaceutical compositions, for instance).

In such cases, therefore, the sample may comprise a mixture of a plurality of source samples, although the source samples may initially be provided separately. Indeed, step (ii) may comprise mixing the source samples together.

A key advantage of combining differing source samples in this way is that the total quantity of target protein present in the sample utilised is increased. This means that the intensity of the m/z peak in the m/z spectrum of the sample is increased, compared to that in the individual source samples. This can enable the detection and analysis of target proteins which would otherwise be indistinguishable from the base noise level in the m/z spectrum of an individual sample. Of course, the multiplexing method is also associated with advantages due to the ease and speed of processing multiple source samples simultaneously.

The number of source samples provided is at least two. For instance, the number of samples provided may be from 2 to 10000, or from 2 to 1000, more usually from 2 to 500 or from 2 to 100.

In the multiplexing process, each chemically differing target protein or, more usually, each differing source sample, is exposed to a polypeptide as described herein. Thus, the sample is exposed to a plurality of polypeptides. As described above, the affinity ligand of each of the polypeptides will bind to each target protein, thus producing a plurality of polypeptide complexes each comprising a polypeptide in complex with a target protein.

Where a single sample comprising a plurality of target proteins is employed, the single sample is exposed to a plurality of polypeptides. Consequently, a first composition comprising a plurality of polypeptide complexes, each comprising a polypeptide (from the said plurality of polypeptides) in complex with a target protein, is directly produced.

More preferably, a plurality of source samples may be used. These may be mixed to form a single sample before being exposed to the plurality of polypeptides. More preferably, though, each individual source sample is exposed to a polypeptide as defined herein, producing a complex comprising the target protein in complex with the said polypeptide. The thus-treated source samples may then be mixed, to form the first composition. This is particularly useful as it allows each source sample to be treated with a chemically different polypeptide. This means that the source sample from which the target protein originated can be determined by determining the identity of the polypeptide with which the target protein is complexed.

Each polypeptide may be the same or different. For instance, each polypeptide within the plurality of polypeptides may comprise the same affinity ligand or a different affinity ligand (different affinity ligands being required where it is intended to bind to differing target proteins). Preferably, each polypeptide within the plurality of polypeptides comprises the same affinity ligand.

The peptide mass tag present in each polypeptide among the plurality of polypeptides may be the same or different.

In a particularly useful embodiment, each source sample is exposed to a different polypeptide. Preferably, each polypeptide in the plurality of polypeptides is isobaric (that is, it has the same weight) and comprises the same affinity ligand, but has a different reporter portion. That is, the arrangement of amino acids within the peptide mass tag differs such that, although each polypeptide is isobaric, the amino acids which constitute the reporter portion as described herein differ. The particular advantage of this embodiment is that the tagged gas-phase ion will have high abundance, as it will contain the isobaric tagged gas-phase ions from each source sample. However, upon fragmentation of the tagged gas-phase ion the differing reporter portions can be used to correlate the target proteins present with their particular source sample.

The reporter portions of each differing polypeptide preferably differ in mass. That is, the reporter portions of each differing polypeptide preferably comprises a non-identical collection of amino acids. That enables the different reporter portion associated with each different polypeptide to be distinguished within a mass spectrum without further fragmentation.

It is alternatively possible for the reporter portions to have the same mass and to differ only in the sequence in which the amino acids therein are ordered. However, this is less preferred as further fragmentation would then be needed to distinguish the sequences of the amino acids present and hence the identity of the reporter ions.

In practice, in this useful embodiment, each source sample is exposed to a plurality of identical polypeptides. However, each source sample is exposed to a plurality of differing polypeptides.

Where it is desired to utilise a plurality of isobaric polypeptides to capture target proteins from differing source samples, and to associate each source sample with a reporter ion of different mass, the maximum number of source samples that can be used will be limited by the number of amino acids present in the peptide mass tag.

For instance, where the peptide mass tag comprises a single amino acid in addition to the other functional portions of the peptide mass tag, that single amino acid may be positioned either in the reporter portion or in the target binding portion. Thus, two permutations of the peptide mass tag having different masses of reporter ion are possible; one containing the additional amino acid and one without. Moreover, if the masses of each of the amino acids in the dipeptide with fragmentation propensity differ from each other and the single additional amino acid present, those amino acids may also be arranged in two ways, leading to two further options for the mass of the reporter portion. Consequently, the maximum number of differing masses of reporter portion and hence the maximum number of source samples which may be employed in the multiplexing process is four in this case.

Where the peptide mass tag comprises two amino acids (of differing masses) in addition to the other functional portions of the peptide mass tag, the reporter portion may comprise neither, either or both of these amino acids. Thus, four different masses of reporter ion are possible. Again, if the mass of each of the amino acids in the dipeptide with fragmentation propensity varies, either of those two amino acids may be present in the reporter portion. This gives rise to eight possible masses of reporter portion. Consequently, the maximum number of source samples which may be employed in the multiplexing process is eight.

With increasing numbers of additional amino acids, the number of masses of reporter ion possible and hence the number of source samples possible increases. For instance, with three available additional amino acids, the number of possible reporter ions is 16; with four additional amino acids, the number of possible reporter ions (and hence source sample possibilities) is 32 (assuming that each additional amino acid employed has a different mass).

Particularly preferably, in this multiplexing method, the plurality of polypeptides is a plurality of polypeptides each comprising an isobaric multiplet of amino acids as described above.

Once the first composition is obtained, it may be treated as described above in connection with steps (iii) to (vi). Accordingly, the multiplexing method described herein involves:
(i) providing
   a sample comprising a plurality of target proteins, and
   a plurality of polypeptides as described herein;
(ii) exposing the sample to the plurality of polypeptides to bind the affinity ligands to the target proteins and thus to produce a first composition comprising a plurality of polypeptide complexes, each polypeptide complex in said plurality comprising a polypeptide in complex with a target protein;
(iii) exposing the first composition to a capture agent, to reversibly bind the polypeptide complexes to the capture agent;
(iv) recovering the polypeptide complexes from the capture agent, to provide a second composition comprising the polypeptide complexes;
(v) ionising the second composition to produce a plurality of tagged gas-phase ions, each tagged gas-phase ion in the said plurality comprising a said polypeptide complex; and
(vi) m/z analysing and detecting the plurality of tagged gas-phase ions, and/or ions derived therefrom.

As noted above, where the sample comprises a plurality of source samples, each of those may be provided separately and exposed to different polypeptides, before being combined in step (ii).

If each polypeptide complex within the plurality of polypeptide complexes is isobaric, then each tagged gas-phase ion will be isobaric. Consequently, when step (v) is performed as above, a single m/z value may be isolated and will comprise the tagged gas-phase ions originating each different source sample.

However, assuming that the plurality of polypeptide complexes comprises such complexes with differing reporter portions, when step (vi) is performed, the plurality of polypeptide complexes will yield a plurality of differing reporter ions. Preferably, as noted above, each differing reporter ion is associated with a differing source sample. Thus, m/z analysing and detecting the reporter ions reveals which sample(s) contained the target protein.

Further, the relative abundance of the differing reporter ions may be indicative of the relative abundance of the target protein in each source sample. Such an abundance analysis may also be carried out on any fragments of the reporter ions produced in a tandem mass spectrometry method as described above, but this can introduce errors (for instance due to differing fragmentation propensities of the reporter ion). Thus, it is preferably to calculate the relative abundance of the target protein in each source sample from the relative abundance of the corresponding reporter ion. The relative abundance of the target protein in any given source sample may be zero, if the target protein is absent from that sample.

Thus, the multiplexing method described herein may comprise identifying the relative quantity of the target protein in each source sample.

### Sample

The sample comprises, at least, a target protein as described herein. The target protein is typically in its native state as in its native membrane environment. That is, any specifically-bound covalent binding partners bound to the membrane protein in its native state remain attached to the target protein. The target protein is not fragmented. For instance, where the target protein comprises multiple sub-units or monomers, the target protein typically comprises all of these sub-units in their native arrangements, rather than one or more fragments thereof. However, minor changes to the native arrangement can be permissible. For instance, a subcomplex of a target protein which is a macromolecular protein assembly is regarded as “intact”, or “native” even if some of the sub-units are not assembled as in the biologically active state.

The sample preferably comprises the target protein in the condition in which it is generally found in its natural biological environment. That is, the sample preferably contains the target protein in the environment in which it is found in nature. Thus, the target protein is preferably in a “wild” state.

The sample is a biological sample, meaning that it comprises material typically found in the human or animal body. The sample is generally a sample which is obtained from a human or animal body, preferably a human body. Often, the sample may be a sample obtained or obtainable from a tissue, biofluid or cell; preferably from a human tissue, biofluid or cell.

However, the sample may alternatively be obtained or obtainable from a non-human or non-animal source, such as a cultured tissue or cell. Thus, the sample may be a harvested sample, harvested from a living body, or a cultured sample, taken from a lab-grown culture.

Examples of samples include samples comprising blood; saliva; a bacterial culture; a collection of cells from, e.g., a biopsy; an entire organ comprised of multiple cell types, e.g., a brain or pancreas; or a tissue culture, e.g., cells grown outside of a living organism.

The cultured or harvested material may be directly utilised in the process. Alternatively, the cultured or harvested material may be subjected to one or more processes in order to produce the sample. For example, cells may be subjected to lysis in order to break cell membranes and release their contents. Lysis processes and other sample manipulations are well-known in the art.

One example of a process performed on the cultured or harvested material to produce a sample is the addition of a solvent. The solvent is typically water, but other solvents may be present (for instance, alcohols such as ethanol or methanol). Accordingly, the sample often comprises one or more solvents (typically water).

The sample usually comprises many other components in addition to the target protein; the biological nature of the sample means that the sample generally contains a complicated mixture of components. For instance, the sample may also contain one or more other proteins; one or more lipids; other peptides (such as RNA) and so on. Consequently, the present process for retrieving the target protein in a form which is suitable for analysis by mass spectrometric methods is most useful.

### Target Protein

The identity of the target protein is not particularly limited. The target protein is typically a native protein, as defined herein.

The target protein may be a membrane protein.

A key aspect of the target protein is that it typically binds to the polypeptide (and specifically the affinity ligand therein) with high efficiency. This enables the polypeptide ligand to recover most, if not all, of the target protein from a sample. For instance, the polypeptide may bind to the target protein with an efficiency of at least 50%, such as at least 60%, 70%, 80%, preferably at least 90%, more preferably at least 95%. By this is meant that, where the polypeptide is mixed with the target protein in equimolar quantities, at least 50% or preferably at least 90% of the polypeptide will bind to the target protein.

The high affinity between the polypeptide and the target protein may also be defined with reference to the dissociation constant (Ka) for the polypeptide complex comprising the target protein and the polypeptide. This complex typically has a Ka of less than 300 nM, more preferably less than 200 nM; further preferably less than 150 nM.

A high affinity between target protein and ligand is particularly important where the target protein is scarce in the sample. If the target protein is highly abundant in the sample, a somewhat lesser affinity between polypeptide and target protein may be acceptable.

### Further Aspects of the Invention

1. A peptide mass tag comprising a dipeptide with fragmentation propensity and a binding moiety.
2. The peptide mass tag according to aspect 1, wherein the dipeptide is selected from D/E-X, I/L/V-X or X-P, wherein X is any amino acid.
3. The peptide mass tag according to aspect 2, wherein the dipeptide is D/E/I/L/V-P, preferably DP.
4. The peptide mass tag according to any one of the preceding aspects, wherein the binding moiety permits purification and elution of a polypeptide comprising the peptide mass tag under non-denaturing conditions.
5. The peptide mass tag according to any one of the preceding aspects, wherein the binding moiety comprises an epitope which reversibly binds to a capture agent, preferably wherein said capture agent comprises an antibody or a peptide ligand.
6. The peptide mass tag according to any one of the preceding aspects, further comprising a proton-donating motif.
7. The peptide mass tag according to aspect 6, wherein the proton-donating motif comprises a polybasic stretch of amino acids.
8. A fusion polypeptide comprising a target polypeptide or an affinity ligand for a target protein fused to a peptide mass tag according to any of the preceding claims.
9. The fusion polypeptide according to aspect 8, wherein the affinity ligand is a peptide ligand, or an antibody.
10. The fusion polypeptide according to aspect 9, wherein the antibody is a single domain antibody or a derivative thereof, preferably a nanobody.
11. The fusion polypeptide according to any of aspects 8 to 10, wherein the affinity ligand has a mass of less than 20 kDa, preferably less than 15 kDa.
12. The fusion polypeptide comprising an affinity ligand for a target protein fused to a peptide mass tag according to any of aspects 8 to 11 which comprises:
   a target-binding portion comprising the affinity ligand, the amino acid of the dipeptide with fragmentation propensity proximal to the affinity ligand, and any intervening amino acid(s); and
   a reporter portion comprising the other amino acid of the dipeptide with fragmentation propensity and the remainder of the peptide mass tag;
      wherein the mass of the reporter portion is less than 6 kDa, and preferably is from 3 kDa to 5 kDa.
13. A composition comprising the fusion polypeptide according to any one of aspects 8-12 in complex with a target protein.
14. A plurality of peptide mass tags according to any one of aspects 1-7 or of fusion polypeptides according to any one of aspects 8-12, wherein each peptide mass tag is isobaric, optionally wherein each fusion polypeptide is isobaric.
15. The plurality of peptide mass tags or fusion polypeptides according to aspect 14, comprising an isobaric multiplet of amino acids comprising the dipeptide with fragmentation propensity, wherein the multiplet varies in sequence in each peptide mass tag.
16. The plurality of peptide mass tags or fusion polypeptides according to aspect 15, wherein the isobaric multiplet of amino acids is at least four amino acids in length.
17. The plurality of fusion polypeptides according to any one of aspects 14 to 16 wherein each fusion polypeptide comprises:
   a target-binding portion comprising an affinity ligand, the amino acid of the dipeptide with fragmentation propensity proximal to the affinity ligand, and any intervening amino acid(s); and
   a reporter portion comprising the other amino acid of the dipeptide with fragmentation propensity and the remainder of the peptide mass tag;

wherein the mass of the reporter portion of each polypeptide is different.
18. A nucleic acid encoding a peptide mass tag according to any one of aspect s 1-7 or a fusion polypeptide according to any one of aspects 8-12, or a plurality of nucleic acids encoding a plurality of peptide mass tags or fusion polypeptides according to any one of aspects 14-17.
19. A vector comprising a nucleic acid according to aspect 18, or a plurality of vectors comprising a plurality of nucleic acids according to aspect 6.
20. A host cell comprising a nucleic acid or plurality of nucleic acids according to aspect 17, or a vector or plurality of vectors according to aspect 18.
21. A kit or library comprising a plurality of fusion polypeptides according to any one of aspects 14-17 or a plurality of nucleic acids or vectors according to aspect 18 or 19.
22. A method of analysing a target protein, the method comprising:
   (i) providing
      a sample comprising the target protein, and
      a polypeptide comprising an affinity ligand for the target protein fused to a peptide mass tag, said peptide mass tag comprising a dipeptide with fragmentation propensity;
   (ii) exposing the sample to the polypeptide to bind the affinity ligand to the target protein and thus to produce a first composition comprising a polypeptide complex comprising the polypeptide in complex with the target protein;
   (iii) exposing the first composition to a capture agent, to reversibly bind the polypeptide complex to the capture agent;
   (iv) recovering the polypeptide complex from the capture agent, to provide a second composition comprising the polypeptide complex;
   (v) ionising the second composition to produce a tagged gas-phase ion, the tagged gas-phase ion comprising the polypeptide complex; and
   (vi) m/z analysing and detecting the tagged gas-phase ion, and/or an ion derived therefrom.
23. The method according to aspect 22 wherein the peptide mass tag comprises a binding moiety, and step (iii) comprises exposing the first composition to the capture agent to reversibly bind the binding moiety to the capture agent.
24. The method according to aspect 22 or aspect 23, wherein the polypeptide is a fusion polypeptide is as defined in any of aspects 8 to 12.

25. The method according to any one of aspects 22 to 24, wherein the sample is obtained or obtainable from a tissue, biofluid or cell.
26. The method according to any one of aspects 22 to 25, wherein the target protein is a native protein.
27. The method according to any one of aspects 22 to 26 wherein the polypeptide binds to the target protein with an efficiency of at least 90%, preferably at least 95%.
28. The method according to any one of aspects 22 to 27 wherein the capture agent comprises a substrate bound to an antibody or antibody derivative or an antigen-binding fragment thereof.
29. The method according to any one of aspects 22 to 28 wherein the capture agent comprises a solid substrate.
30. The method according to any one of aspects 22 to 29 wherein step (vi) comprises:
   (vi)(a) subjecting the tagged gas-phase ion to an ion transformative process to produce a first-generation product ion; and
   (vi)(b) m/z selecting and optionally detecting the first-generation product ion.
31. The method according to aspect 30 wherein the polypeptide complex comprises:
   a target-binding portion comprising the target protein, the affinity ligand, the amino acid of the dipeptide with fragmentation propensity bound to the affinity ligand, and any intervening amino acid(s); and
   a reporter portion comprising the other amino acid of the dipeptide with fragmentation propensity and the remainder of the peptide mass tag;

and the first-generation product ion produced in step (vi)(a) comprises the reporter portion.
32. The method according to aspect 30 or aspect 31 which comprises:
   (vi)(c) subjecting the first-generation product ion to a further ion transformative process to produce a second generation product ion; and
   (vi)(d) m/z analysing and optionally detecting the second generation product ion.
33. The method according to any one of aspects 30 to 32 wherein each ion transformative process is selected from infrared multiphoton dissociation, electron transfer dissociation, activated ion electron transfer dissociation, electron capture dissociation, UV photodissociation and collision-induced dissociation.
34. The method according to any one of aspects 22 to 33 wherein the method comprises:
   (i) providing
      a sample comprising a plurality of target proteins, and
      a plurality of polypeptides comprising an affinity ligand for the target protein fused to a peptide mass tag, said peptide mass tag comprising a dipeptide with fragmentation propensity, optionally being a plurality of fusion polypeptides as defined in any one of aspects 14 to 17;
   (ii) exposing the sample to the plurality of polypeptides to bind the affinity ligands to the target proteins and thus to produce a first composition comprising a plurality of polypeptide complexes, each polypeptide complex in said plurality comprising a polypeptide in complex with a target protein;
   (iii) exposing the first composition to a capture agent, to reversibly bind the polypeptide complexes to the capture agent;
   (iv) recovering the polypeptide complexes from the capture agent, to provide a second composition comprising the polypeptide complexes;
   (v) ionising the second composition to produce a plurality of tagged gas-phase ions, each tagged gas-phase ion in the said plurality comprising a said polypeptide complex; and
   (vi) m/z analysing and detecting the plurality of tagged gas-phase ions, and/or ions derived therefrom.
35. The method according to aspect 34, wherein the sample comprises a mixture of a plurality of source samples, wherein each source sample is obtained or obtainable from a tissue, biofluid or cell.
36. The method according to aspect 35, wherein each source sample is exposed to a different polypeptide among the plurality of polypeptides.
37. The method according to aspect 35 or aspect 36, wherein the method comprises identifying the relative quantity of the target protein in each source sample.

### EXAMPLES

### Materials and Methods

### Nanobody Expression and Purification

Sequences of VHH domains were obtained from literature^{xii} and custom genes encoding the VHH with the designer modular domains were synthesized (Integrated DNA technologies). Genes encoding the designer affinity capture reagents, including a PelB leader sequence and C-terminal custom TDT tag, were cloned into pet vectors using ligation independent cloning (Takara Biosciences). Plasmids isolated from single colonies were sequenced using Sanger Sequencing. Several colonies from overnight growth following transformation of plasmids into BL21 Lemo cells were picked and grown overnight in LB broth supplemented with ampicillin. ˜20 mL of the overnight culture was used to inoculate 1L of LB broth supplemented with ampicillin, and grown at 37° C. (120 RPM) until an OD600˜0.6 was reached. Cultures were then supplemented with 0.5 mM IPTG grow overnight at 16° C. before harvesting the cells by centrifugation at 8,000×g for 30 minutes. Pelleted cells were resuspended in TES buffer and allowed to incubate for 2 hours in 4×-dilute TES buffer to induce outer membrane rupture. The nAbs were purified from periplasmic extracts using Ni-NTA affinity chromatography, concentrated to ˜1 mg/mL, and then snap frozen in LN2 before storage at −80° C.

### Lysozyme Purification

Egg whites were diluted 4× with water and stirred with a magnetic stirrer at 4° C. for 4 hours to precipitate ovomucin.^{xviii} The mixture was centrifuged at 10,000×g for 10 minutes and the supernatant was loaded into a dialysis cassette (10 kDa MWCO) for dialysis overnight into 20 mM HEPES pH 8.0, 50 mM NaCl. After overnight dialysis, the solution was centrifuged at 10,000×g for 10 minutes to pellet additional precipitated protein and the supernatant was filtered using a syringe through a 0.45 μM filter. The concentration of the diluted egg white mixture was determined by absorbance at 280 nm and determined to have an approximate overall protein concentration of 20 mg/mL. Approximately 300 μg of nanobody was added to a 1 mL aliquot of the protein mixture and imidazole pH 8.0 was added to 20 mM before incubation at 4° C. for 1 hour. A mini spin column containing 50 μL of Ni-NTA resin was equilibrated by several washes with equilibration buffer (20 mM HEPES pH 8.0, 50 mM NaCl, 20 mM imidazole pH 8.0). After the 1 hour incubation, the ˜1 mL of nanobody-lysozyme mixture was added to the resin and centrifuged at 700×g for 1 minute and the column was washed 3 additional times (700×g, 1 minute) with the equilibration buffer. The nanobody-lysozyme complex was eluted from the resin twice with 100 μL of elution buffer (20 mM HEPES pH 8.0, 50 mM NaCl, 350 mM imidazole pH 8.0) and centrifuged at 700×g for 1 minute. The eluted protein was concentrated using a centrifugal concentrator (30 kDa MWCO) and the protein concentration was monitored by absorbance measurements at 280 nm.

### Native Mass Spectrometry

Native mass spectrometry was performed on a QExactive Ultra High Mass Resolution (UHMR) Orbitrap instrument. 1-3 μL of buffer-exchanged protein or protein complex was loaded into a gold-coated glass capillary (1.2 mm O.D.) pulled to a fine tip. A voltage of 1.1 to 1.2 kV was applied to the capillary to generate an electrospray which was directed into the mass spectrometer through a transfer capillary heated to 100° C. Following transfer into the instrument, in-source activation (10 V) was applied to aid in desolvation. For intact mass measurements, ions were detected in the Orbitrap at a resolving power of 12,500 (@ m/z 200). For tandem mass spectrometry (MS²) experiments, ions were first isolated in the quadrupole with an isolation window of 20 m/z. Dissociation of noncovalent interactions was achieved using 100 V of activation in the HCD cell, and backbone fragmentation of the protein was achieved using 150 V of activation. Fragments or dissociated subunits were then directed into the Orbitrap for detection with 100 ms inject times. MS² spectra were collected at a resolving power of 200,000 (@ m/z 200) and averaged for 100 scans.

### Example 1—Overview of Design of Affinity Ligand with Peptide Mass Tag

We designed a suite of nanobodies (nAbs) with modular domains, making them uniquely suitable for affinity capture and protein identification by native mass spectrometry and tandem mass spectrometry, respectively (FIG. 1A). The modular domains consist of: (1) a VHH domain that binds with high affinity to the protein target, (2) a pair of amino acids with high propensity for gas-phase fragmentation, (3) a multiplet of amino acids flanking the cleavable domain which give rise to a fragment ion of unique mass (reporter ion), (4) an epitope tag that can be used to reversibly affix the nanobody to a solid support for affinity purification strategies, and, (5) a string of basic amino acids which effectively donate additional protons that are needed to promote backbone fragmentation at the desired site. Domains 2 through 5 represent a novel “top-down tag” (TDT).

We recorded native mass spectra of the designer nanobody in FIG. 1A and observed a series of well-resolved charge states spanning the 8-6′ species (FIG. 1B, top). These charge states deconvolute to the expected molecular weight of 17,967.5±0.4 Da, and no signs of non-physiological dimerization can be observed. To demonstrate the ability for the TDT to be liberated from the intact nAb, we isolated the 8 charge state of the intact protein (FIG. 1B, middle) and subjected it to energetic collisions to induce backbone fragmentation; the resulting tandem MS spectrum was populated by product ions which corresponded to different backbone scission events along the proteins' primary structure (FIG. 1B, bottom).

### Domain 2: the Amino Acid Pair for Efficient Gas-Phase Fragmentation

Domain 2 contains a carefully placed amino acid pair known for highly predictable and specific fragmentation^{xiii}, in this case, breaking the amide bond between the aspartic acid (D) and proline (P). Therefore, along with the many possible product ions that are generated from cleavage of amino acid bonds along the nAb-TDT (FIG. 1B, bottom), product ions resulting from D|P cleavage consisting of the amino acid sequence PIFMDYKDDDDKASGENLYFQSLHHHHHH (SEQ ID NO: 7) should be produced. Inspection of the tandem MS spectra in the bottom panel of FIG. 1B, showed that there were two highly abundant fragment ions that corresponded to this amino acid sequence-those of the singly protonated species at 3533 m/z (FIG. 1B, right panel), and the doubly protonated at 1767 m/z. Importantly, the resulting singly charged ion at 3533 m/z was in a region of m/z spectra that is typically sparsely populated by product ions in tandem MS experiments. The expected monoisotopic mass of this ion is 3532.56 Da, which is identical to the measured mass. Therefore, this ion is useful and reliable as a distinct peak that serves as a unique “reporter ion” for the nanobody (or nanobody-target complex).

### Domain 5: The Polybasic Stretch of Amino Acid Residues

We anticipated that singly and doubly protonated product ions for the TDT would be present. However, we were surprised to observe that the singly protonated species was present in high abundance. Protons adducted to proteins, which allow for their detection as molecular ions, are also responsible for driving backbone fragmentation during tandem MS experiments. Thus, we engineered a polybasic site nearby the susceptible D|P bond to ensure protons were available and could be sufficiently “donated” to direct backbone cleavage at the preferred site to liberate the diagnostic fragment(s). Use of hexa-histidine as the stretch of basic residues simultaneously serves as a proton sink to sequester H⁺ ions for mobile proton directed peptide fragmentation, and also serves as a useful purification handle during the isolation of the designer nAb from E coli supernatants. The polybasic site representing domain 5 is likely to be advantageous in various cases, as historically, native electrospray conditions result in a low overall charge density for protein complexes, which results in poor fragmentation efficiency for such intact proteins.^{xiv}

### Domain 1: Affinity Ligand

We investigated the use of an nAb as an exemplary affinity ligand. Over the last 20 years, single domain antibodies from camelids (e.g., nanobodies or VHHs) have been identified as novel affinity capture reagents, as their small size and high affinity (K_{d}S˜100 nM or less) binding to antigens makes them especially suitable for downstream biophysical techniques (e.g. FRET spectroscopy, cryoelectron tomography). In addition, their limited repertoire of post-translational modifications and the ability to generate large quantities using bacterial expression systems make them highly amenable to MS.^{xv,xvi} The Chait lab has designed nAbs for generating structural models of protein complexes using peptide-centric mass spectrometry (e.g., LC-MS/proteomics, crosslinking MS).^{xv,xvi} nAbs have been used for affinity capture coupled to LC-MS, and have undergone many iterations of protein design to limit cross-reactivity with chemical crosslinkers making them very powerful tools for peptide-based MS. However, such downstream methods effectively destroy the non-covalent and non-crosslinkable interactions (e.g. small molecules bound to proteins), and therefore have limited utility in native MS studies focused on capturing the diversity of protein-molecule interactions directly from endogenous sources.

It was therefore attractive to develop a suite of similar tools that enables for high-affinity antigen binding that is compatible with native MS.

The nAb in FIG. 1B has a molecular weight of 17,967.5±0.4 Da, and it is anticipated that substitution of Domain 1 with VHHs targeting different endogenous proteins will result in intact masses of 13 to 18 kDa, depending on the unique VHH sequence for an individual protein target. As the designer nanobodies are small, soluble proteins that are expressed in bacterial expression systems, they can be made in milligram quantities and will contain limited post-translational modifications. As a result of this expression strategy, the native mass spectrum of the nAb in FIG. 1B was of low complexity, had few adduct peaks adjacent the main charge state distribution (which dilutes signal intensities across many m/z peaks), and the main charge state signals were highly resolved (˜1-2 Th at FWHM). This limited spectral complexity for these high-affinity capture reagents means that the intact mass determined by deconvolution of the charge states in the native mass spectrum is extremely accurate. The simple, low molecular weight nAb is in stark contrast with conventional monoclonal antibodies used in immunoprecipitations, which weigh in excess of 150 kDa and are heavily N-and O-glycosylated which distribute signals across many regions of the m/z spectral space and skews mass accuracy.

### Domain 3: Residues Flanking Domain 2 Enable Sample Multiplexing

In the context of Domain 3, the ability to generate an affinity capture reagent and characterize its mass with extremely high accuracy is highly significant. In this regard, consider the organization of amino acid residues that flank either side of the labile amino acid pair (Domain 2). In the design presented in FIG. 1A, they have the sequence AAADPIFM (SEQ ID NO: 23), where Domain 2 is underlined. By swapping a residue preceding Domain 3 for one following, e.g., IAADPAFM (SEQ ID NO: 24), a new nAb with a different primary structure but exactly the same molecular mass (isobars) will be generated.

Domain 1 remains unchanged, so this new nAb sequence will be endowed with the same high-affinity binding as that in FIG. 1A. And, these two reagents will share the same molecular weight (since they are comprised of the same amino acids), however, the resulting diagnostic fragments produced by tandem MS scission at the labile Domain 2 dipeptide bond will have different m/z values. Three of such amino acid sequences for Domain 3 are presented in FIG. 1C-each nAb complex produced will have molecular weight 17,967 Da, however, the diagnostic fragments in FIG. 1C will have average masses of 3534.78, 3492.70, and 3458.69 Da. Singly charged product ions will therefore be measured at 3535.79, 3592.71, and 3459.70 m/z, respectively. These product ions are distinct, and therefore allow for the presence of each of the three nAbs within a mixture to be determined with high confidence. Furthermore, if the three reagents in FIG. 1C are used to capture protein antigens from separate samples (e.g., patient tissue, biofluids) and subsequently pooled, their intensities relative to one another will report on the relative quantities of the antigens which they captured. In other words, the position of amino acids in Domain 3 allows for antigen-nab complexes to be multiplexed into a single measurement—removing potential experimental error associated with individual measurements and aggregation of results. This is described in more detail below.

In addition, the number of unique diagnostic fragments which can be made are a direct result of the multiplet of amino acids in domain 3; the amino acids can be rearranged such that several nanobodies can be made with identical (isobaric) intact molecular mass, but during tandem MS analysis, dissociate to yield unique peptide fragments with different masses. The triplet design described here allows for the generation of a suite of nanobodies which yield eight unique reporter TDTs for multiplexing applications. In practice, the multiplet of amino acids can be increased to a greater number of amino acids, including unnatural amino acids or covalently modified amino acids, which will result in a greater number of nanobodies harboring unique TDTs for multiplexing.

### Example 2—Proof of Concept of nAb-Protein Complex Formation

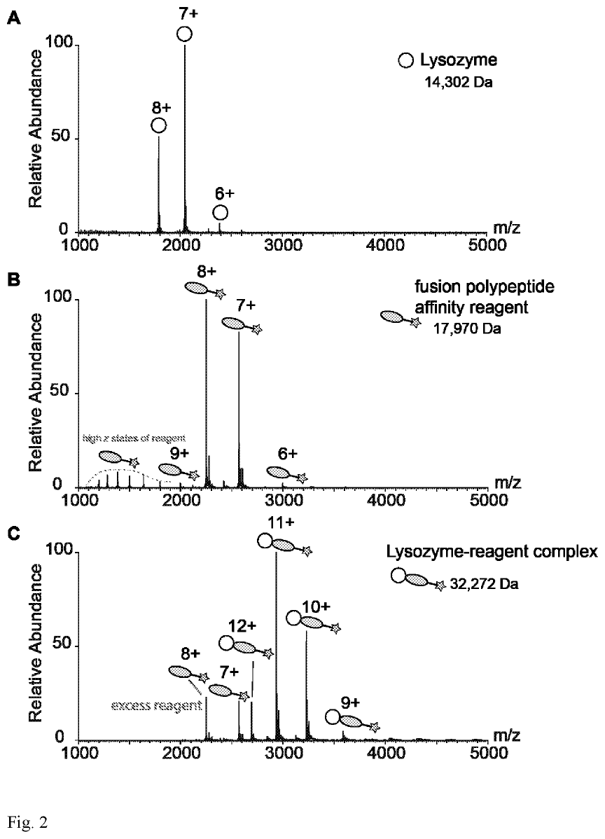

Nanobodies recognize structural components of their antigens and display a high level of shape complementarity.^{xvii} To demonstrate that the additional domains did not inhibit the conformation dependent VHH from binding the target protein, we designed a suite of designer nanobodies with a VHH domain specific to hen egg white lysozyme. In a native mass spectrum, protonated ions of lysozyme (MW=14,302 Da) are distributed across an m/z range of 1788 to 2384 m/z, corresponding to charge states 8+ to 6+, respectively (FIG. 2A). The designer nanobody weighs 17,967.5 Da, where the modular domains 2 through 5 make up 4,121 Da of the total molecular weight. In the mass spectrum, ions corresponding to the unbound nanobody are centered at the 8+ charge state at m/z 2247 (FIG. 2B). When mixed in approximately equimolar ratio, we observed 1:1 binding of the nanobody-lysozyme complex with a measured molecular weight of 32,272±2 Da (FIG. 2C), demonstrating that the addition of the modular domains appended to the C-terminus of the VHH did not inhibit antigen binding.

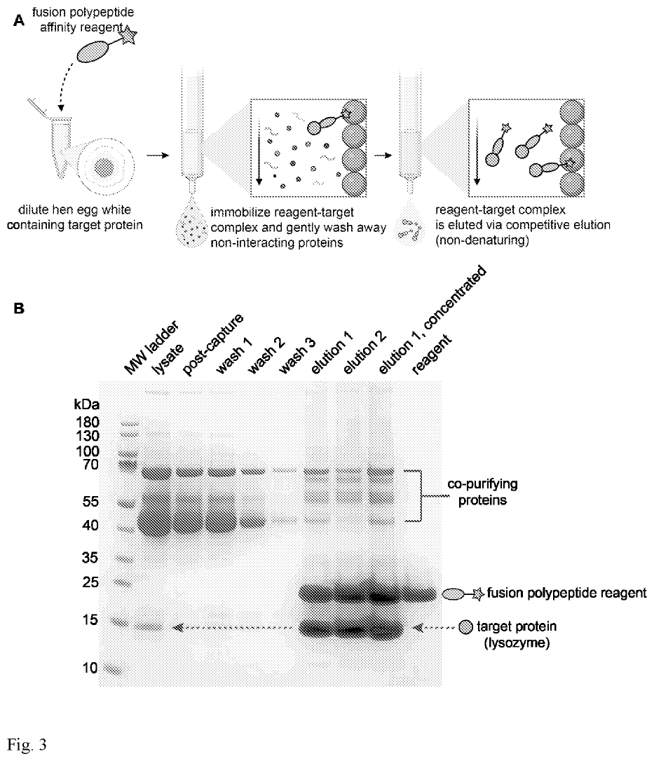

The cartoon depiction of the nanobody is similar to that of a fishing lure, as it is an apt representation for its purpose; the designer nanobody is first used to “fish” target proteins from an endogenous source (e.g. cell lysates, tissue homogenates, or biofluids) prior to mass spectrometry and subsequent top-down mass spectrometry analyses. As a primary goal of nMS is identify non-covalent interactions between proteins and ligands, the nAb-extracted protein target, while still in complex with the nAb, is gently eluted from the affinity resin for analysis (FIG. 3A). As the nAbs are small, they can be left on the protein targets, with no detriment to the native MS analyses. To demonstrate a proof-of-concept study, the protein lysozyme was extracted from its endogenous source (hen egg white) using the nAb strategy depicted in FIG. 3A. In a dilute solution of hen egg white, lysozyme is expected to constitute ˜3% of the total protein concentration,^{xviii} which was reflected by the low intensity band of a lysate of hen egg whites detected by Coomassie Brilliant Blue staining following SDS-PAGE (FIG. 3B). The band at ˜15 kDa corresponding to lysozyme was not present in the post-capture, indicating that it had been successfully bound to the immobilized nAb. This band was also not present during the washing steps used to clear the immobilized nAb-antigen complex of non-specifically bound proteins. In the successive competitive (and non-denaturing) elutions, the target protein and nAbs were evident. Furthermore, the thick, dark band for the 15 kDa species indicated that the targeted lysozyme had been efficiently enriched from its native environment. This proof of concept indicates that the designer nanobodies can efficiently capture target endogenous proteins from complex mixtures.

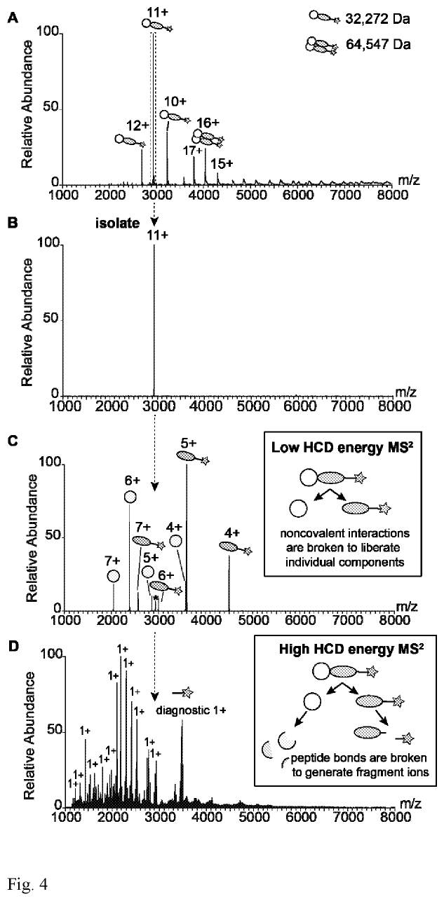

The nanobody-target complex was the most abundant peak series in the mass spectrum, centered at the 11+ charge state at m/z 2934 (FIG. 4A). Adjacent to this series, we observed a distribution of non-specific nanobody-target dimers, which is a direct result of the high concentration of target protein captured. A low abundance continuum of signals were observed beyond m/z 4500, attributed to additional co-purifying species resulting from the gentle extraction of lysozyme with a minimal number of handling steps.

To confirm the identity of the major peak series, we isolated the 11+ charge state at m/z 2934, and subjected the ions to activation using higher-energy collisional dissociation

(HCD). At relatively at low activation energies (˜100 V), non-covalent interactions were broken and the nanobody dissociated from the target protein (FIG. 4B). At sufficiently high activation (˜150 V), bond dissociation occurred and peptide fragments originating from the nanobody and target protein were observed (FIG. 4C). The MS² fragment spectrum resulting from higher energy dissociation was populated by abundant singly charged ions; those with highest intensity were attributed to fragments originating from the modular domains of the designer nanobody and the remaining low m/z ions were assigned to fragment ions of lysozyme (Data Not Shown). The abundant fragment ion at m/z 3534 corresponded to the 1+ charge state of the unique TDT (or the y₂₉ fragment of the designer nanobody), while the remaining high intensity ions between 2200 and 2600 m/z corresponded to high propensity DID cleavage of the epitope tag included in domain 4. Despite the strong non-covalent interaction with the target protein, and overall lower charge density of the complex (relative to the individual components), the designer TDT was still efficiently liberated with high intensity in an MS2 experiment and remained well separated in the m/z spectrum from the remainder of the fragment ions.

### Example 3—Multiplex Capability of the Peptide Mass Tag

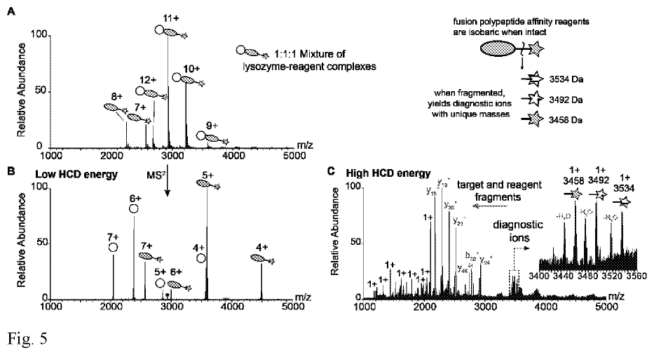

To demonstrate the ability to multiplex the designer nanobodies without the detriment of complicated MS¹ spectra, we made an approximately equimolar mixture of three designer nanobodies in complex with the target protein and analyzed the mixture by native mass spectrometry. The MS¹ spectrum revealed two charge state distributions: a small population of unbound nanobody which was centered at the 7⁺ charge state, and a high abundance distribution corresponding to the nanobody-target protein complex centered at the 11+ charge state at m/z 2934 (FIG. 5A). The use of low activation energy (˜100 V) broke the non-covalent interactions between the nanobody and the target and liberated the individual components. As the designer nanobodies contained the same overall amino acid sequence, they were isobaric and indistinguishable from one another in the MS¹ and low-energy MS² spectrum (FIG. 5B). Under conditions of high activation (˜150 V), fragmentation of both the nanobody and lysozyme occurred, revealing an MS² spectrum that was densely populated with singly and doubly charged fragment ions originating from both protein components. Notably, we observed singly charged ions which result from the intentional fragmentation of the D|P bond, liberating the unique TDT reporter ions that were well resolved from one another at m/z's 3458, 3492, and 3534 (FIG. 5C). The intensities of the reporter ions were roughly equivalent, indicating that relative quantitation of the protein complex could be retained in intensity information of the unique fragment.

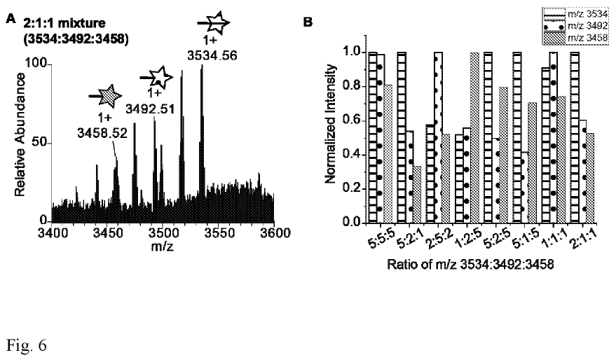

To determine if relative protein concentration was retained in the reporter ion intensities, we analyzed a series of mixtures containing different ratios of three isobaric nanobody-target complexes. For a mixture containing a molar ratio of 2:1:1 nanobody-lysozyme complexes, the mass spectrum revealed a single charge state distribution, as the masses of each intact complex were identical and resulted in no discernible difference in m/z. Upon fragmentation via deposition of high activation energy, we generated a spectrum populated with fragment ions, including the well-resolved singly charged reporter ions derived from each unique nanobody (FIG. 6A). Indeed, the intensities of each reporter ion reflected the relative abundance of each complex in the mixture, as reporter ion at m/z 3534 was nearly double the intensity of the reporter ions at m/z 3492 and 3458, consistent with the molarity of the components of the original mixture. From the raw intensities of the isotopic envelope of each reporter ion, we calculated a relative ratio of 1.9:1.1:1.0 of the nanobody-lysozyme complexes. Several other ratios of nanobody-lysozyme complexes were tested, and the raw intensities of reporter ions consistently reflected the concentration of the original mixture (FIG. 6B).

### Example 4—Demonstration of the Capacity of the Affinity Capture Reagents to Capture Proteins From Tissue Sections

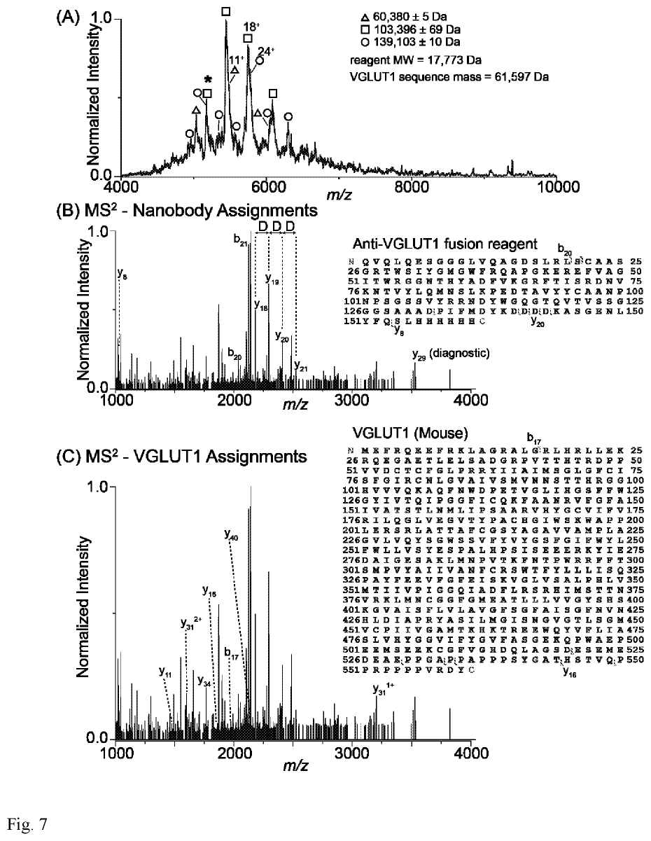

To demonstrate that an affinity capture reagent was capable of capturing a targeted protein (the vesicular glutamate transporter 1, VGLUT1) directly from a tissue section, a single mouse brain was lysed in tris-buffered saline solution containing a non-ionic detergent. The lysate was clarified of cellular debris by centrifugation (10,000×g, 10 min) and subsequently mixed with an affinity capture reagent (a nanobody targeting VGLUT1) immobilized on an affinity chromatography resin. After incubation, the lysate was separated from the magnetic particles containing the immobilized affinity capture reagent and any captured VGLUT1. The resin was washed with buffer to clear any non-specifically associated proteins prior to incubation with buffer containing a peptide (DYKDDDDK) (SEQ ID NO: 10) to competitively elute the bound affinity capture reagents, including those associated with VGLUT1. The native mass spectrum in FIG. 7A was generated by electrospray ionisation of the eluate. Molecular weights for the most abundant charge state distributions are listed in the inset, with corresponding peak assignments indicated with symbols. The anticipated molecular weight for the affinity capture reagent and for wild VGLUT1 (with no post-translational or post-transcriptional processing considered) are also listed. A peak, labelled with an asterisk, belonged to two of the charge state distributions which corresponded to molecular weights consistent with free VGLUT1 and dimeric VGLUT1 bound to an affinity capture reagent. This peak was subjected to tandem MS analysis: it was m/z isolated and subjected to higher-energy collision induced dissociation (HCD) to generate amino acid fragment ions from the intact protein ions. The resulting tandem MS spectrum was searched against the amino acid sequence for the affinity capture reagent (FIG. 7B). Several theoretical fragment ions matched the peaks in the experimental spectrum, including the diagnostic ion corresponding to the cleavage at the D/P moiety in the polypeptide (y₂₉ in FIG. 7B). The tandem MS spectrum was also searched for theoretical fragment ions from wild-type murine VGLUT1, without considering any post-transcriptional or post-translational processing (FIG. 7C). Labelled on the spectrum are the experimental fragment ions that matched the theoretical following a manual inspection of each peak for isotopic spacing. Sequential fragment ions are matched at the C-terminus provided evidence for the assignment of this peak as VGLUT1 with the nAb bound. Therefore, we demonstrated that the affinity capture reagents are capable of isolating wild-type proteins directly from tissue sections.

### Example 5—Demonstration of Reproducibility and the Vapacity of the Affinity Capture Reagents for Co-Immunoprecipitation Studies From Tissue Sections

As a final example, and to demonstrate reproducibility between tissue sections, VGLUT1 was captured from different mouse brain (distinct from that used in Example 4) following lysis in solution comprised of tris-buffer saline and a non-ionic detergent. The homogenate was processed identically to that described in Example 4. An affinity capture reagent (a nanobody targeting VGLUT1) immobilised onto anti-DYKDDDDK (SEQ ID NO:10) magnetic agarose was incubated with the homogenate, washed of non-specifically associated proteins/molecules, and the affinity capture reagent along with any non-covalently bound/associated protein(s) were released from the resin via competitive elution with DYKDDDDK (SEQ ID NO:10) peptide. The eluate was analysed by native mass spectrometry (FIG. 8A). The native mass spectrum was populated by two charge state distributions of near equal intensity—one broad distribution with diffuse peaks (>100 m/z wide) centred at a 19+ charge state. The deconvolved molecular mass, determined using the apex of each adjacent peaks, was found to be 82,904±114 Da. This molecular weight is in agreement with monomeric VGLUT1 (˜61 kDa) bound to a single affinity capture reagent (17 kDa). The other charge state distribution, centred at the 23+ charge state, corresponded to a deconvolved molecular mass of 131,699 kDa. HCD was used to generate fragment ions from the peak labelled as the 19+ charge state consistent with VGLUT1 bound to a single affinity capture reagent (FIG. 8B). The diagnostic y₂₉ fragment ion resulting from cleavage at the D/P polypeptide was identified and is labelled within the inset. Additional fragment ions corresponding to anticipated cleavage along the peptide mass tag (DYKDDDK (SEQ ID NO:10) in FIG. 8B) were identified. Several additional fragment ion peaks, which did not correspond to the affinity capture reagent, were observed. A database search of these peaks against theoretical peaks that are anticipated to originate from mouse VGLUT1 was carried out. Many more peaks could be assigned as fragment ions originating from mouse VGLUT1, which are mapped onto the sequence in FIG. 8C. This provides direct evidence that the affinity capture reagents can reproducibly capture a target protein from different biological samples. Finally, the 23+ charge state (the peak near 5700 m/z labelled with black diamond) was fragmented by HCD via tandem MS. The resulting tandem MS spectrum was absent of any fragment ions characteristic of the invention, indicating that no affinity capture reagents were bound (FIG. 8D). An open database search against the entire mouse proteome was carried out to identify the protein(s) that co-immunopurified with VGLUT1 and the affinity capture reagent. The search results provided matches of several of the peaks in the spectrum to fragment ions originating from GrpE1 (FIG. 8E). GrpE1 is a 23 kDa protein, so this must be in complex with other proteins to account for the ˜123 kDa measured in FIG. 8A.

### Discussion of Results

For extraction of target proteins from endogenous sources, nanobodies offer several advantages to conventional monoclonal antibodies. For one, mAbs are highly glycosylated, which makes their analysis complicated as the heterogeneity in glycan structure results in many different masses, which produce broad and overlapping signals in native mass spectra. This dilutes the signal across many different m/z peaks, which reduces the signal-to-noise ratio to a point which complicates collecting useful interpretable native mass spectra of low abundance mAb-target protein complexes. Taking advantage of the low molecular weight and simple composition of the nAbs, they can remain bound during native MS experiments with no obvious detriment to the analysis of the target protein. By engineering the domain (3) to have a high propensity to fragment during tandem mass spectrometry experiments, a liberated fragment is designed to serve as a “reporter” ion for the nAb-target protein complex, which circumvents the low propensity for gas-phase fragmentation of natively folded proteins. This makes it feasible to carry out molecular identification under conditions where fragment ion generation is limiting.

The reporter ions generated by the genetically-encoded sequence tags are akin to those found in isobaric labelling through tandem mass tags (TMT) proteomics workflows, where mixtures of proteins originating from different samples can be quantified based on isotope modifications and intensities. Our method however does not rely on chemical modifications and already exceeds the level of multiplexing offered by commercial TMT and isobaric labeling approaches. Continued development and optimization of the reporter tags will increase the library of available designer nAbs. For one, the dipeptide in domain 2 is variable and can be replaced by any other pair of amino acids with high fragmentation propensity (e.g. any D/E|X, I/L/V|X, or X|P motif), see also the dipeptides described in Haverland et al infra^{xiii}, incorporated herein by reference. In addition, increasing the number of amino acids to the reporter fragment and/or adding a reactive amino acid to the reporter ion (such as a cysteine) allows for the coupling of any number of molecules that would have distinct molecular weights, without significantly altering the fragmentation propensity. With these strategies, it is feasible to achieve multiplexing of >24 samples using designer nAbs.

Here, we demonstrate proof-of-concept using lysozyme as a model antigen, as it has been used extensively to lay the groundwork for understanding of antigen-paratope binding^{xix} and also serves as a model protein for native and top-down mass spectrometry experiments.^{xx,xxi} We have successfully demonstrated the workflow is useful for capturing membrane-embedded receptors and transporters from individual brain tissue sections from mice and other animals. Animal to animal reproducibility was demonstrated, indicating that that the workflow and affinity capture reagents have the capacity to analyse targeted proteins from clinical (patient) specimens. Even difficult-to-fragment proteins such as proteins with low charge density, few basic residues, or proteins containing extensive disulphide bonding can be unambiguously identified by the unique reporter ions liberated from the designer nAb. Furthermore, low-abundance proteins can now be pooled and analyzed with no loss of information about the original source; we envision applications (i) in clinical cohorts where patient to patient comparisons (e.g. healthy versus disease) are essential, (ii) in differential tissue expression analysis (i.e. how a protein differs across tissue or cell types), and (iii) as a means to track target proteins within mixtures following a molecular perturbation (i.e. how the intact mass of the target protein changes relative to its post-translational modifications or its interactions with other molecules). The demonstration that co-immunoprecipitating proteins can be retained provides further evidence that delicate interactions, even those too transient or delicate to be maintained as stable complexes in the mass spectrometer, can be discovered.

### REFERENCES

ⁱ Chen, S.; Getter, T.; Salom, D.; Wu, D.; Quetschlich, D.; Chorev, D. S.; Palczewskim K.; Robinson, C. V. Capturing a rhodopsin receptor signalling cascade across a native membrane. Nature. 2022, 604, 384-390.
ⁱⁱ Su, C-C.; Lyu, M.; Morgan, C. E.; Bolla, J. R.; Robinson, C. V.; Yu, E. W. A ‘Build and Retrieve’ methodology to simultaneously solve cryo-EM structures of membrane proteins. Nat. Methods. 2021, 18, 69-75.
ⁱⁱⁱ Ye, Y.; Wu, H.; Chen, K.; Clapier, C. R.; Verma, N.; Zhang, W.; Deng, H.; Cairns, B. R.; Gao, N.; Chen, Z. Structure of the RSC complex bound to the nucleosome. Science. 2019, 366, 838-843.
^{iv} Silvester, E.; Vollmer, B.; Prazak, V.; Vasishtan, D.; Machala, E. A.; Whittle, C.; Black, S.; Bath, J.; Turberfield, A. J.; Grunewald, K.; Baker, L. A. DNA origami signposts for identifying proteins on cell membrane by electron tomography. Cell. 2021, 184, 1110-1121.e16.
^{v} Zhao, J.; Makhija, S.; Zhou, C.; Zhang, H.; Wang, Y-Q.; Muralidharan, M.; Huang, B.; Cheng, Y. Structural insights into the human PA28-20S proteasome enabled by efficient tagging and purification of endogenous proteins. Proc. Natl. Acad. Sci. U. S. A. 2022, doi: 10.1073/pnas.2207200119.
^{vi} Lantz, C.; Zenaidee, M. A.; Wei, B.; Hemminger, Z.; Ogorzalek Loo, R. R.; Loo, J. A. ClipsMS: An Algorithm for Analyzing Internal Fragments Resulting from Top-Down Mass Spectrometry. J. Prot. Res. 2021, 20, 1928-1935.
^{vii} Wei, B.; Zenaidee, M. A.; Lantz, C.; Ogorzalek Loo, R. R.; Loo, J. A. Towards understanding the formation of internal fragments generated by collisionally activated dissociation for top-down mass spectrometry. Anal. Chim. Acta. 2022, doi: 10.1016/j.aca.2021.339400.
^{viii} Liko, I.; Allison, T. M.; Hopper, J. T. S.; Robinson, C. V. Mass spectrometry guided structural biology. Curr. Opin. Struct. Biol. 2016, 40, 136-144.
^{ix} Gault, J.; Liko, I.; Landreh, M.; Shutin, D.; Bolla, J. R.; Jefferies, D.; Agasid, M.; Yen, H-Y.; Ladds, M. J. G. W.; Lane, D. P.; Khalid, S.; Mullen, C.; Remes, P. M.; Huguet, R.; McAlister, G.; Goodwin, M.; Viner, R.; Syka, J. E. P.; Robinson, C. V. Combining native and ‘omics’ mass spectrometry to identify endogenous ligands bound to membrane proteins. Nat. Methods. 2020, 17, 505-508.
^{x} Fornelli, L.; Toby, T. K. Characterization of large intact protein ions by mass spectrometry: What directions should we follow? Biochim. Biophys. Acta. Proteins Proteom. 2022, doi.org/10.1016/j.bbapap.2022.140758
^{xi} Wysocki, V. H.; Tsaprailis, G.; Smith, L. L.; Breci, L. A. Mobile and localized protons: a framework for understanding peptide dissociation. J. Mass Spectrom. 2000, 35, 1399-1406.
^{xii} Akiba, H.; Tamura, H.; Kiyoshi, M.; Yanaka, S.; Sugase, K.; Caaveiro, J. M. M.; Tsumoto, K. Structural and thermodynamic basis for the recognition of the substrate-binding cleft on hen egg lysozyme by a single-domain antibody. Sci Rep. 2019, 9, 15481. doi: 10.1038/s41598-019-50722-y.
^{xiii} Haverland, N. A.; Skinner, O. S.; Fellers, R. T.; Tariq, A. A.; Early, B. P.; LeDuc, R. D.; Fornelli, L.; Compton, P. D.; Kelleher, N.L. Defining Gas-Phase Fragmentation Propensities of Intact Proteins During Native Top-Down Mass Spectrometry. J. Am. Soc. Mass Spectrom. 2017, 28, 1203-1215.
^{xiv} Zhou, M.; Lantz, C.; Brown, K. A.; Ge, Y.; Pasa-Tolic, L.; Loo, J. A.; Lermyte, F. Higher-order structural characterisation of native proteins and complexes by top-down mass spectrometry. Chem. Sci. 2020, 11, 12918-12936.
^{xv} Fridy, P. C.; Li, Y.; Keegan, S.; Thompson, M. K.; Nudelman I.; Scheid, J. F.; Oeffinger, M.; Nussenzweig, M. C.; Fenyo, D.; Chait, B. T.; Rout, M. P. A robust pipeline for rapid production of versatile nanobody repertoires. Nat. Methods. 2014, 11, 1253-1260.
^{xvi} Shi, Y.; Pellarin, R.; Fridy, P. C.; Fernandez-Martinez, J.; Thompson, M. K.; Li, Y.; Wang, Q-J.; Sali, A.; Rout, M. P.; Chait, B. T. A strategy for dissecting the architectures of native macromolecular assemblies. Nat. Methods. 2015, 12, 1135-1138.
^{xvii} Mitchell, L. S.; Colwell, L. J. Comparative analysis of nanobody sequence and structure data. Proteins. 2018, 86, 697-706.
^{xviii} Omana, D. A.; Wu, J. A New Method of Separating Ovomucin from Egg White. J. Agric. Food Chem. 2009, 57, 3596-3603.
^{xix} De Genst, E.; Silence, K.; Decanniere, K.; Conrath, K.; Loris, R.; Kinne, J.; Muyldermans, S.; Wyns, L. Molecular basis for the preferential cleft recognition by dromedary heavy-chain antibodies. Proc. Natl. Acad. U. S. A. 2006, 103, 4586-4591.
^{xx} Quick, M. M; Crittenden, C. M.; Rosenberg, J. A.; Brodbelt, J. S. Characterization of Disulfide Linkages in Proteins by 193 nm Ultraviolet Photodissociation (UVPD) Mass Spectrometry. Anal. Chem. 2018, 90, 8523-8530.
^{xxi} Wei, B.; Zenaidee, M. A.; Lantz, C.; Williams, B.; Totten, S.; Ogorzalek Loo, R. R.; Loo, J. A. Top-Down Mass Spectrometry and Assigning Internal Fragments for Determining Disulfide Bond Positions in Proteins. ChemRxiv. DOI: 10.26434/chemrxiv-2022-65k4h
^{xxii} Zimmermann I, Egloff P, Hutter C A J, Kuhn B T, Bräuer P, Newstead S, Dawson R J P, Geertsma E R, Seeger M A. Generation of synthetic nanobodies against delicate proteins. Nat Protoc. 2020 May; 15 (5): 1707-1741. doi: 10.1038/s41596-020-0304-x. Epub 2020 Apr. 8. PMID: 32269381.
^{xxiii} Iwan Zimmermann, Pascal Egloff, Cedric A J Hutter, Fabian M Arnold, Peter Stohler, Nicolas Bocquet, Melanie N Hug, Sylwia Huber, Martin Siegrist, Lisa Hetemann, Jennifer Gera. Samira Gmür, Peter Spies, Daniel Gygax, Eric R Geertsma, Roger J P Dawson, Markus A Seeger (2018) Synthetic single domain antibodies for the conformational trapping of membrane proteins eLife 7: e34317.

1. A peptide mass tag comprising a dipeptide with fragmentation propensity and a binding moiety.

2. The peptide mass tag according to claim 1, wherein the dipeptide is selected from D/E-X, I/L/V-X, X-P or F-W, wherein X is any amino acid.

3. The peptide mass tag according to claim 2, wherein the dipeptide is D/E/I/L/V-P, preferably DP.

4. The peptide mass tag according to claim 1, wherein the binding moiety permits purification and elution of a polypeptide comprising the peptide mass tag under non-denaturing conditions.

5. The peptide mass tag according to claim 1, wherein the binding moiety comprises an epitope which reversibly binds to a capture agent, or wherein said capture agent comprises an antibody or a peptide ligand.

6. The peptide mass tag according to claim 1, further comprising a proton-donating motif.

7. The peptide mass tag according to claim 6, wherein the proton-donating motif comprises a polybasic stretch of amino acids.

8. A fusion polypeptide comprising a target polypeptide or an affinity ligand for a target protein fused to a peptide mass tag according to claim 1.

9. The fusion polypeptide according to claim 8, wherein the affinity ligand is a peptide ligand, or an antibody.

10. The fusion polypeptide according to claim 9, wherein the antibody is a single domain antibody or a derivative thereof, or a nanobody.

11. The fusion polypeptide according to claim 8, wherein the affinity ligand has a mass of less than 20 kDa, preferably less than 15 kDa.

12. The fusion polypeptide comprising an affinity ligand for a target protein fused to a peptide mass tag according to claim 8 which comprises:
a target-binding portion comprising the affinity ligand, the amino acid of the dipeptide with fragmentation propensity proximal to the affinity ligand, and any intervening amino acid(s); and
a reporter portion comprising the other amino acid of the dipeptide with fragmentation propensity and the remainder of the peptide mass tag;
wherein the mass of the reporter portion is less than 6 kDa, or is from 3 kDa to 5 kDa.

13. A composition comprising the fusion polypeptide according to claim 8 in complex with a target protein.

14. A plurality of peptide mass tags according to claim 1, or of fusion polypeptides comprising a target polypeptide or an affinity ligand for a target protein fused to a peptide mass tag according to claim 1, wherein each peptide mass tag is isobaric, optionally wherein each fusion polypeptide is isobaric.

15. The plurality of peptide mass tags or fusion polypeptides according to claim 14, comprising an isobaric multiplet of amino acids comprising the dipeptide with fragmentation propensity, wherein the multiplet varies in sequence in each peptide mass tag.

16. The plurality of peptide mass tags or fusion polypeptides according to claim 15, wherein the isobaric multiplet of amino acids is at least four amino acids in length.

17. The plurality of fusion polypeptides according to claim 16 wherein each fusion polypeptide comprises:
a target-binding portion comprising an affinity ligand, the amino acid of the dipeptide with fragmentation propensity proximal to the affinity ligand, and any intervening amino acid(s); and
a reporter portion comprising the other amino acid of the dipeptide with fragmentation propensity and the remainder of the peptide mass tag;
wherein the mass of the reporter portion of each polypeptide is different.

18. A nucleic acid encoding a peptide mass tag according to claim 1 or a fusion polypeptide comprising a target polypeptide or an affinity ligand for a target protein fused to a peptide mass tag according to claim 1.

19. A vector comprising a nucleic acid according to claim 18.

20. A host cell comprising: a nucleic acid or plurality of nucleic acids encoding a peptide mass tag according to claim 1 or a fusion polypeptide comprising a target polypeptide or an affinity ligand for a target protein fused to a peptide mass tag according to claim 1; or a vector or plurality of vectors comprising a nucleic acid encoding a peptide mass tag according to claim 1.

21. A kit or library comprising:
a plurality of fusion polypeptides comprising a target polypeptide or an affinity ligand for a target protein fused to a peptide mass tag according to claim 1, wherein each peptide mass tag is isobaric, optionally wherein each fusion polypeptide is isobaric; or a plurality of nucleic acids each encoding a peptide mass tag according to claim 1, or a fusion polypeptide comprising a target polypeptide or an affinity ligand for a target protein fused to a peptide mass tag according to claim 1; or
a plurality of vectors comprising said nucleic acid.

22. A method of analysing a target protein, the method comprising:
(i) providing
a sample comprising the target protein, and
a polypeptide comprising an affinity ligand for the target protein fused to a peptide mass tag, said peptide mass tag comprising a dipeptide with fragmentation propensity;
(ii) exposing the sample to the polypeptide to bind the affinity ligand to the target protein and thus to produce a first composition comprising a polypeptide complex comprising the polypeptide in complex with the target protein;
(iii) exposing the first composition to a capture agent, to reversibly bind the polypeptide complex to the capture agent;
(iv) recovering the polypeptide complex from the capture agent, to provide a second composition comprising the polypeptide complex;
(v) ionising the second composition to produce a tagged gas-phase ion, the tagged gas-phase ion comprising the polypeptide complex; and
(vi) m/z analysing and detecting the tagged gas-phase ion, and/or an ion derived therefrom.

23-25. (canceled)


SEQUENCE LISTING (27 sequences)

SEQ ID NO: 1 - protein, 37 aa, synthetic construct
GGGSAAADPIFMDYKDDDDKASGENLYFQSLHHHHHH

SEQ ID NO: 2 - protein, 37 aa, synthetic construct
GGGSIAADPAFMDYKDDDDKASGENLYFQSLHHHHHH

SEQ ID NO: 3 - protein, 37 aa, synthetic construct
GGGSFAADPIAMDYKDDDDKASGENLYFQSLHHHHHH

SEQ ID NO: 4 - protein, 8 aa, synthetic construct
GGGSAAAD

SEQ ID NO: 5 - protein, 8 aa, synthetic construct
GGGSIAAD

SEQ ID NO: 6 - protein, 8 aa, synthetic construct
GGGSFAAD

SEQ ID NO: 7 - protein, 29 aa, synthetic construct
PIFMDYKDDDDKASGENLYFQSLHHHHHH

SEQ ID NO: 8 - protein, 29 aa, synthetic construct
PAFMDYKDDDDKASGENLYFQSLHHHHHH

SEQ ID NO: 9 - protein, 29 aa, synthetic construct
PIAMDYKDDDDKASGENLYFQSLHHHHHH

SEQ ID NO: 10 - protein, 8 aa, synthetic construct
DYKDDDDK

SEQ ID NO: 11 - protein, 10 aa, synthetic construct
EQKLISEEDL

SEQ ID NO: 12 - protein, 9 aa, synthetic construct
TETSQVAPA

SEQ ID NO: 13 - protein, 9 aa, synthetic construct
YPYDVPDYA

SEQ ID NO: 14 - protein, 4 aa, synthetic construct
SGSG

SEQ ID NO: 15 - protein, 6 aa, synthetic construct
SGSGSG

SEQ ID NO: 16 - protein, 8 aa, synthetic construct
SGSGSGSG

SEQ ID NO: 17 - protein, 10 aa, synthetic construct
SGSGSGSGSG

SEQ ID NO: 18 - protein, 16 aa, synthetic construct
SGSGSGSGSGSGSGSG

SEQ ID NO: 19 - protein, 161 aa, synthetic construct
QVQLQESGGGLVQAGDSLRLSCAASGRTWSIYGMGWFRQAPGKEREFVAGITWRGGNTHYADFVKGRFTISRDNVKNTVYLQMNSLKPEDTAVYYCAANPNPSGSSVYRRNDYWGQGTQVTVSSGGGSAAADPIFMDYKDDDDKASGENLYFQSLHHHHHH

SEQ ID NO: 20 - protein, 560 aa, Mus musculus
MEFRQEEFRKLAGRALGRLHRLLEKRQEGAETLELSADGRPVTTHTRDPPVVDCTCFGLPRRYIIAIMSGLGFCISFGIRCNLGVAIVSMVNNSTTHRGGHVVVQKAQFNWDPETVGLIHGSFFWGYIVTQIPGGFICQKFAANRVFGFAIVATSTLNMLIPSAARVHYGCVIFVRILQGLVEGVTYPACHGIWSKWAPPLERSRLATTAFCGSYAGAVVAMPLAGVLVQYSGWSSVFYVYGSFGIFWYLFWLLVSYESPALHPSISEEERKYIEDAIGESAKLMNPVTKFNTPWRRFFTSMPVYAIIVANFCRSWTFYLLLISQPAYFEEVFGFEISKVGLVSALPHLVMTIIVPIGGQIADFLRSRHIMSTTNVRKLMNCGGFGMEATLLLVVGYSHSKGVAISFLVLAVGFSGFAISGFNVNHLDIAPRYASILMGISNGVGTLSGMVCPIIVGAMTKHKTREEWQYVFLIASLVHYGGVIFYGVFASGEKQPWAEPEEMSEEKCGFVGHDQLAGSDESEMEDEAEPPGAPPAPPPSYGATHSTVQPPRPPPPVRDY

SEQ ID NO: 21 - protein, 6 aa, synthetic construct
ENLYFQ

SEQ ID NO: 22 - protein, 8 aa, synthetic construct
LEVLFQGP

SEQ ID NO: 23 - protein, 8 aa, synthetic construct
AAADPIFM

SEQ ID NO: 24 - protein, 8 aa, synthetic construct
IAADPAFM

SEQ ID NO: 25 - protein, 100 aa, synthetic construct
  MOD_RES 1: note = N-acetylmethionine
MEFRQEEFRKLAGRALGRLHRLLEKRQEGAETLELSADGRPVTTHTRDPPVVDCTCFGLPRRYIIAIMSGLGFCISFGIRCNLGVAIVSMVNNSTTHRGG

SEQ ID NO: 26 - protein, 60 aa, synthetic construct
EEMSEEKCGFVGHDQLAGSDESEMEDEAEPPGAPPAPPPSYGATHSTVQPPRPPPPVRDY

SEQ ID NO: 27 - protein, 217 aa, Mus musculus
MAARCVRLARRSLPALALSFRPSPRLLCTATKQKNNGQNLDEDLGHCEPKTDPPSADKTLLEEKAKLEEQLRETMEKYKRALADTENLRQRSQKLVEEAKLYGIQGFCKDLLEVADILEKATQSVPKEEISNNNPHLKSLYEGLVMTEVQIQKVFTKHGLLRLDPIGAKFDPYEHEALFHTPVEGKEPGTVALVSKVGYKLHGRTLRPALVGVVKDA